(19) United States
(12) Patent Application Publication
(10) Pub. No.: US 2012/0022400 A1
(43) Pub. Date: Jan. 26, 2012

(54) Biopsy Device with Detachable Needle Tip Protector

(76) Inventor: Daniel J. Mumaw, Milford, OH (US)

(21) Appl. No.: 12/843,088

(22) Filed: Jul. 26, 2010

(51) Int. Cl. A61B 10/02

(52) U.S. Cl. 600/567

(57) ABSTRACT

A biopsy device comprises a body, a needle, a cutter, and a needle tip protector. The needle tip protector is selectively attachable to the biopsy device before and/or after a procedure, to protect a user from contact with the sharp needle and to prevent the needle from damage during handling. In some versions, the needle tip protector may be placed on, and removed from, the biopsy device with the user's hands remaining proximal from the distal end of the needle. In some versions, the needle tip protector substantially seals the lateral aperture of the needle such that the needle can be primed before a procedure. In some versions, the needle tip protector includes one or more living hinges for selectively securing the needle tip protector to the biopsy device; or for actuating the needle tip protector to shield or expose the distal end of the needle.

### BACKGROUND

Biopsy samples have been obtained in a variety of ways in various medical procedures using a variety of devices. Biopsy devices may be used under stereotactic guidance, ultrasound guidance, MRI guidance, PEM guidance, BSGI guidance, or otherwise. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

Merely exemplary biopsy devices are disclosed in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pub. No. 2003/0109803, entitled “MRI Compatible Surgical Biopsy Device,” published Jun. 12, 2003; U.S. Pub. No. 2006/0074345, entitled “Biopsy Apparatus and Method,” published Apr. 6, 2006; U.S. Pub. No. 2007/0118048, entitled “Remote Thumbwheel for a Surgical Biopsy Device,” published May 24, 2007; U.S. Pub. No. 2008/0214955, entitled “Presentation of Biopsy Sample by Biopsy Device,” published Sep. 4, 2008; U.S. Pub. No. 2009/0171242, entitled “Clutch and Valving System for Tetherless Biopsy Device,” published Jul. 2, 2009; U.S. Non-Provisional patent application Ser. No. 12/335,578, entitled “Hand Actuated Tetherless Biopsy Device with Pistol Grip,” filed Dec. 16, 2008; U.S. Non-Provisional patent application Ser. No. 12/337,942, entitled “Biopsy Device with Central Thumbwheel,” filed Dec. 18, 2008; U.S. Non-Provisional patent application Ser. No. 12/483,305, entitled “Tetherless Biopsy Device with Reusable Portion,” filed Jun. 12, 2009; and U.S. Non-Provisional patent application Ser. No. 12/709,624, entitled “Spring Loaded Biopsy Device,” filed Feb. 22, 2010. The disclosure of each of the above-cited U.S. patents, U.S. Patent Application Publications, and U.S. Non-Provisional Patent Applications is incorporated by reference herein.

While several systems and methods have been made and used for obtaining a biopsy sample, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of an exemplary biopsy device.

FIG. 2 depicts a block schematic view of components that are part of, or used with, the device of FIG. 1.

FIG. 3 depicts a first series view of part of the needle of the biopsy device of FIG. 1, with the needle shown in cross section and with the cutter in the initial, distal position.

FIG. 4 depicts a second series view of part of the needle of the biopsy device of FIG. 1, with the needle shown in cross section and with the cutter in an intermediate position during retraction.

FIG. 5 depicts a third series view of part of the needle of the biopsy device of FIG. 1, with the needle shown in cross section and with the cutter in the retracted, proximal position.

FIG. 6 depicts a fourth series view of part of the needle of the biopsy device of FIG. 1, with the needle shown in cross section and with the cutter in the advanced, distal position.

FIG. 7 depicts a perspective view of an exemplary needle tip protector for use with the device of FIG. 1.

FIG. 8 depicts a cross section view of the needle tip protector of FIG. 7.

FIG. 9 depicts a front view of another exemplary needle tip protector for use with the device of FIG. 1, shown with the needle tip protector in a closed position.

FIG. 10 depicts a front view of the needle tip protector of FIG. 9, shown with the needle tip protector in an open position.

FIG. 11 depicts a top view of another exemplary needle tip protector for use with the device of FIG. 1, shown with the needle tip protector covering the needle tip.

FIG. 12 depicts a top view of the needle tip protector of FIG. 11, shown with the needle tip protector extended distally, exposing the needle tip.

FIG. 13 depicts a side view of the distal end of another exemplary needle tip protector with a portion of the distal end of the needle tip protector shown in cross section, and with portions of the needle shown in phantom.

FIG. 14 depicts a top view of the needle tip protector of FIG. 13, with the needle shown in phantom.

FIG. 15 depicts a bottom view of the needle tip protector of FIG. 14, with portions of the needle shown in phantom.

FIG. 16 depicts a top view of another exemplary needle tip protector for use with the device of FIG. 1, with some components or portions of components shown in phantom.

FIG. 17 depicts a side view of the needle tip protector of FIG. 16, with some components or portions of components shown in phantom.

FIG. 18 depicts a top view of another exemplary needle tip protector for use with the device of FIG. 1.

FIG. 19 depicts a top view of the needle tip protector of FIG. 18, shown with the needle tip protector in cross section.

FIG. 20 depicts an exemplary alternate distal end for the needle tip protector of FIG. 18.

FIG. 21 depicts another exemplary alternate distal end for the needle tip protector of FIG. 18.

FIG. 22 depicts another exemplary alternate distal end for the needle tip protector of FIG. 18.

FIG. 23 depicts a top view of another exemplary needle tip protector for use with the device of FIG. 1, shown with the living hinges in the closed position.

FIG. 24 depicts a top view of the needle tip protector of FIG. 23, shown with the living hinges in the open position.

FIG. 25 depicts a top view of the needle tip protector of FIG. 23, with the body and boots of the needle tip protector shown in cross section and a portion of a needle inserted within the needle tip protector.

FIG. 26 depicts a perspective view of another exemplary needle tip protector for use with the device of FIG. 1.

FIG. 27 depicts an enlarged perspective view of the proximal portion of the needle tip protector of FIG. 26.

FIG. 28 depicts an end view of an exemplary needle hub for use with the needle tip protector of FIG. 26.

FIG. 29 depicts a cross section view of the needle hub of FIG. 28, taken along line 29-29 of FIG. 28.

FIG. 30 depicts a top view of another exemplary needle tip protector for use with the device of FIG. 1, shown in cross section.

FIG. 31 depicts a top view of another exemplary needle tip protector for use with the device of FIG. 1, shown in cross section with the needle tip protector in the open position.

FIG. 32 depicts a top view of the needle tip protector of FIG. 31, shown in cross section with the needle tip protector in the closed position.

FIG. 33 depicts a side view of another exemplary needle tip protector for use with the device of FIG. 1, shown with the elongated needle hub retracted within the housing of the body of the biopsy device.

FIG. 34 depicts a side view of the needle tip protector of FIG. 33, shown with the elongated needle hub extended from the housing of the biopsy device.

FIG. 35 depicts a top view of another exemplary needle tip protector for use with the device of FIG. 1, shown with the shield component of the housing of the biopsy device retracted.

FIG. 36 depicts a top view of the needle tip protector of FIG. 35, shown with the shield component of the housing of the biopsy device extended.

FIG. 37 depicts a side view of the needle tip protector of FIG. 36.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

I. Overview

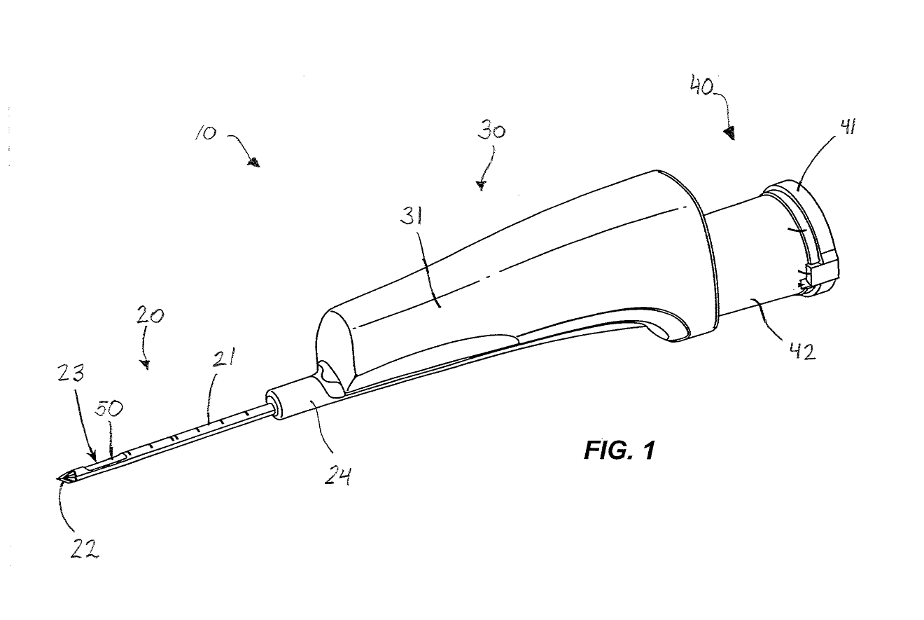

As shown in FIG. 1, an exemplary biopsy device (10) comprises a needle (20), a body (30), a tissue sample holder (40), and a cutter (50). In particular, needle (20) extends distally from the distal portion of body (30), while tissue sample holder (40) extends proximally from the proximal portion of body (30). Body (30) is sized and configured such that biopsy device (10) may be operated by a single hand of a user. In particular, a user may grasp body (30), insert needle (20) into a patient's breast, and collect one or a plurality of tissue samples from within the patient's breast, all with just using a single hand. Alternatively, a user may grasp body (30) with more than one hand and/or with any desired assistance. In some settings, the user may capture a plurality of tissue samples with just a single insertion of needle (20) into the patient's breast. Such tissue samples may be pneumatically deposited in tissue sample holder (40), and later retrieved from tissue sample holder (40) for analysis. While examples described herein often refer to the acquisition of biopsy samples from a patient's breast, it should be understood that biopsy device (10) may be used in a variety of other procedures for a variety of other purposes and in a variety of other parts of a patient's anatomy.

Needle (20) of the present example comprises a cannula (21) with a tissue piercing tip (22), a lateral aperture (23), and a hub (24). Tissue piercing tip (22) is configured to pierce and penetrate tissue, without requiring a high amount of force, and without requiring an opening to be pre-formed in the tissue prior to insertion of tip (22). Alternatively, tip (22) may be blunt (e.g., rounded, flat, etc.) if desired. Lateral aperture (23) is sized to receive a tissue from a tissue specimen during operation of device (10). Within cannula (21) resides cutter (50), which rotates and translates relative to cannula (21) and past lateral aperture (23) to sever a tissue sample from tissue protruding through lateral aperture (23). Hub (24) may be formed of plastic that is overmolded about needle (20) or otherwise secured to needle (20), such that hub (24) is unitarily secured to needle (20). Alternatively, hub (24) may be formed of any other suitable material through any suitable process and may have any other suitable relationship with needle (20). Hub (24) of the present example is coupled with a vacuum conduit (not shown), and is operable to communicate a vacuum (or atmospheric air, saline, pressurized fluid, etc.) from vacuum conduit to lateral aperture (23). The vacuum conduit may be coupled with a variety of sources, including but not limited to a vacuum source that is internal or external to biopsy device (10) in accordance with the teachings of U.S. Non-Provisional patent application Ser. No. 12/483,305, entitled “Tetherless Biopsy Device with Reusable Portion,” filed Jun. 12, 2009, and/or U.S. Pub. No. 2008/0214955, entitled “Presentation of Biopsy Sample by Biopsy Device,” published Sep. 4, 2008, the disclosures of which are incorporated by reference herein. Still other suitable fluid sources that a vacuum conduit may be coupled with will be apparent to those of ordinary skill in the art in view of the teachings herein. Of course, any suitable type of valve(s) and/or switching mechanism(s) may also be coupled with vacuum conduit, e.g., as taught in U.S. Non-Provisional patent application Ser. No. 12/483,305, entitled “Tetherless Biopsy Device with Reusable Portion,” filed Jun. 12, 2009, and/or U.S. Pub. No. 2008/0214955, entitled “Presentation of Biopsy Sample by Biopsy Device,” published Sep. 4, 2008, the disclosures of which are incorporated by reference herein. It should also be understood that a vacuum, atmospheric air, a liquid such as saline, etc. may also be selectively communicated to the lumen defined by cutter (50).

Body (30) of the present example comprises a housing (31). In some versions, body (30) is formed in at least two pieces, comprising a probe portion and a holster portion. For instance, in some such versions, the probe portion may be separable from the holster portion. Furthermore, the probe portion may be provided as a disposable component while the holster portion may be provided as a reusable portion. By way of example only, such a probe and holster configuration may be provided in accordance with the teachings of U.S. Non-Provisional patent application Ser. No. 12/483,305, entitled “Tetherless Biopsy Device with Reusable Portion,” filed Jun. 12, 2009, and/or U.S. Pub. No. 2008/0214955, entitled “Presentation of Biopsy Sample by Biopsy Device,” published Sep. 4, 2008, the disclosures of which are incorporated by reference herein. Alternatively, any other suitable probe and holster configuration may be used. It should also be understood that body (30) may be configured such that it does not have a separable probe portion and holster portion. Various other suitable ways in which body (30) may be configured will be apparent to those of ordinary skill in the art in view of the teachings herein.

Tissue sample holder (40) of the present example comprises a cap (41) and an outer cup (42). A filter tray (not shown) is provided within outer cup (42). Outer cup (42) is secured to body (30) in the present example. Such engagement may be provided in any suitable fashion. Outer cup (42) of the present example is substantially transparent, allowing the user to view tissue samples on the filter tray, though outer cup (42) may have any other suitable properties if desired. The hollow interior of outer cup (42) is in fluid communication with cutter (50) and with a vacuum source in the present example. By way of example only, vacuum may be provided to outer cup (42), and such a vacuum may be further communicated to cutter (50), in accordance with the teachings of U.S. Non-Provisional patent application Ser. No. 12/483,305, entitled “Tetherless Biopsy Device with Reusable Portion,” filed Jun. 12, 2009, and/or U.S. Pub. No. 2008/0214955, entitled “Presentation of Biopsy Sample by Biopsy Device,” published Sep. 4, 2008, the disclosures of which are incorporated by reference herein. Various other suitable ways in which vacuum may be provided to outer cup (42) will be apparent to those of ordinary skill in the art in view of the teachings herein. It should also be understood that outer cup (42) may receive vacuum from the same vacuum source as the vacuum conduit in needle (20). Biopsy device (10) may further include one or more valves (e.g., shuttle valve, electromechanical solenoid valve, etc.) to selectively regulate communication of a vacuum and/or other fluids to outer cup (42) and/or vacuum conduit, regardless of whether outer cup (42) and vacuum conduit are coupled with a common source of vacuum or other source of fluid.

In the present example, when a tissue sample has been severed from a tissue specimen by cutter (50), the tissue sample is pulled from cutter (50) to tissue sampler holder (40) by the vacuum. Cap (41) is removably coupled with outer cup (42) in the present example such that a user may remove cap (41) to access tissue samples that have gathered on the filter tray (not shown) within outer cup (42) during a biopsy process. In lieu of having a stationary filter tray, tissue sample holder (40) may have a plurality of trays that are removably coupled with a rotatable manifold, such that the manifold is operable to successively index each tray relative to cutter (50) to separately receive tissue samples obtained in successive cutting strokes of cutter (50). For instance, tissue sample holder (40) may be constructed and operable in accordance with the teachings of U.S. Pub. No. 2008/0214955, entitled “Presentation of Biopsy Sample by Biopsy Device,” published Sep. 4, 2008, the disclosure of which is incorporated by reference herein. As another merely illustrative example, tissue sample holder (40) may be constructed and operable in accordance with the teachings of U.S. Non-Provisional patent application Ser. No. 12/337,911, entitled “Biopsy Device with Discrete Tissue Chambers,” filed Dec. 18, 2008. Still other suitable ways in which tissue sample holder (40) may be constructed and operable will be apparent to those of ordinary skill in the art in view of the teachings herein.

It should be understood that, as with other components described herein, needle (20), body (30), tissue sample holder (40), and cutter (50) may be varied, modified, substituted, or supplemented in a variety of ways, and that needle (20), body (30), tissue sample holder (40), and cutter (50) may have a variety of alternative features, components, configurations, and functionalities. Several merely exemplary variations, modifications, substitutions, or supplementations are described in U.S. Non-Provisional patent application Ser. No. 12/709,624, entitled “Spring Loaded Biopsy Device,” filed Feb. 22, 2010, the disclosure of which is hereby incorporated by reference. Still yet, other suitable alternative versions, features, components, configurations, and functionalities of needle (20), body (30), tissue sample holder (40), and cutter (50) will be apparent to those of ordinary skill in the art in view of the teachings herein.

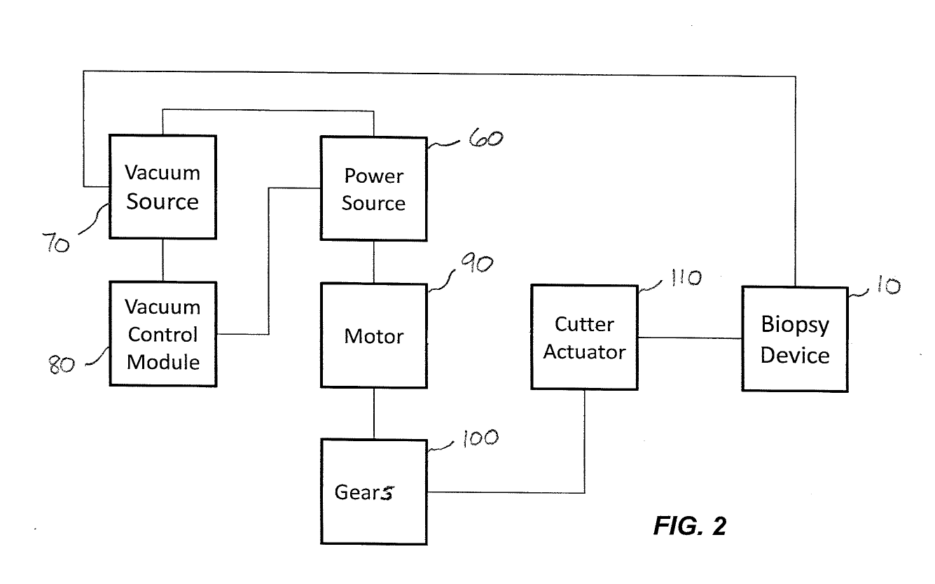

As shown in FIG. 2, exemplary components that are part of, or used with, the device of FIG. 1, some of which have been introduced above, include a power source (60), a vacuum source (70), a vacuum control module (80), a motor (90), a set of gears (100), and a cutter actuator (110). In the present example, power source (60) provides power to vacuum source (70), vacuum control module (80), and motor (90). In some versions, power source (60) is located onboard biopsy device (10), e.g., a battery; while in some other versions, power source (60) is located some distance from biopsy device (10), e.g., line voltage from a standard electrical receptacle with a cable connection to biopsy device (10) and/or through an additional module between an electrical receptacle and biopsy device (10). Various configurations for and modifications to power source (60) will be apparent to those of ordinary skill in the art in view of the teachings herein.

In the present example, vacuum source (70) provides vacuum to biopsy device (10) for drawing tissue into lateral aperture (23) of needle (20). Vacuum source (70) also provides vacuum to biopsy device (10) for transporting a severed tissue sample from cutter (50) to tissue sample holder (40). In some versions, vacuum source (70) comprises a vacuum pump located onboard biopsy device (10). By way of example only, such an onboard vacuum source (70) may comprise a diaphragm pump that is driven by motor (90). In some such versions, vacuum source (70) is not coupled with power source (60) and vacuum control module (80) is omitted. In some other versions, vacuum source (70) comprises a vacuum pump located some distance from biopsy device (10) that provides vacuum via a vacuum cable or conduit. Of course, vacuum source (70) may comprise a combination of a vacuum pump located within housing (31) and a vacuum pump that is external to housing (31), if desired. In the present example, vacuum source (70) is in communication with vacuum control module (80). Vacuum control module (80) includes functions to control the supply and delivery of vacuum from vacuum source (70) to biopsy device (10). Various functions and capabilities that can be used with vacuum control module (80) to control how vacuum is supplied and delivered will be apparent to those of ordinary skill in the art in view of the teachings herein. Also, various other configurations for, and modifications to, vacuum source (70) and vacuum control module (80) will be apparent to those of ordinary skill in the art based on the teachings herein.

Motor (90) of the present example comprises a conventional DC motor, though it should be understood that any other suitable type of motor may be used. By way of example only, motor (90) may comprise a pneumatic motor (e.g., having an impeller, etc.) that is powered by pressurized air, a pneumatic linear actuator, an electromechanical linear actuator, a piezoelectric motor (e.g., for use in MRI settings), or a variety of other types of movement-inducing devices. As mentioned above, motor (90) receives power from power source (60). In some versions, motor (90) is located onboard biopsy device (10) (e.g., within housing (31)). In some other versions, motor (90) is located some distance from biopsy device (10) and provides energy to biopsy device (10) via a drive shaft or cable. In the present example, motor (90) is operable to rotate a drive shaft (not shown), which extends distally from motor (90) to gear set (100) to provide a rotary input into gear set (100). While the drive shaft extends directly from motor (90) into gear set (100), it should be understood that a variety of other components may be coupled between motor (90) and gear set (100), including but not limited to various gears, a clutch, etc. Gear set (100) includes an output shaft (not shown) having a drive gear (not shown) secured thereto, and is operable to selectively activate cutter actuator (110). Gear set (100) may comprise a planetary gearbox, and may be configured to provide speed reduction. Various suitable configurations for motor (90) and gear set (100) will be apparent to those of ordinary skill in the art in view of the teachings herein.

Cutter actuator (110) of the present example comprises a variety of components that interact to provide simultaneous rotation and distal translation of cutter (50) relative to body (30) and needle (20) in a firing stroke. Cutter actuator (110) is also operable to retract cutter (50) proximally to ready cutter (50) for firing. By way of example only, cutter actuator (110) may be configured and operable in accordance with the teachings of U.S. Non-Provisional patent application Ser. No. 12/709,624, entitled “Spring Loaded Biopsy Device,” filed Feb. 22, 2010, and/or U.S. Pub. No. 2008/0214955, entitled “Presentation of Biopsy Sample by Biopsy Device,” published Sep. 4, 2008, the disclosures of which are incorporated by reference herein. It should be understood that, as with other components described herein, cutter actuator (110) may be varied, modified, substituted, or supplemented in a variety of ways, and that cutter actuator (110) may have a variety of alternative features, components, configurations, and functionalities. Suitable alternative versions, features, components, configurations, and functionalities of cutter actuator (110) will be apparent to those of ordinary skill in the art in view of the teachings herein.

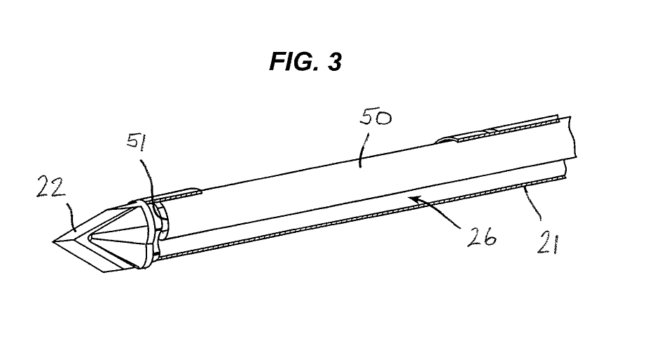

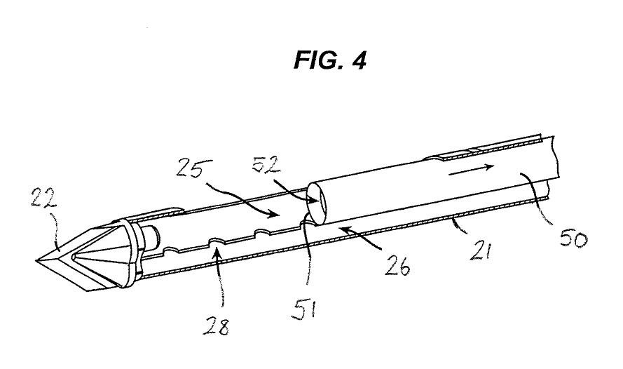

As shown in the series views of FIGS. 3-6, an exemplary cutter (50) firing sequence is shown. FIG. 3 depicts cutter (50) in a distal position, with distal edge (51) of cutter (50) positioned distal of lateral aperture (23) thereby effectively “closing” lateral aperture (23) of needle (20). In this configuration, needle (20) can be inserted without tissue prolapsing through lateral aperture (23). FIG. 4 depicts cutter (50) being retracted by cutter actuator (110), thereby exposing tissue to lateral aperture (23) and revealing a cutter lumen (52) of cutter (50). In the present example, cutter (50) is positioned within a first lumen (25) of cannula (21). Beneath first lumen (25) is a second lumen (26), which is in part defined by a divider (27). Divider (27) comprises a plurality of openings (28) that provide fluid communication between first and second lumens (25, 26). A plurality of external openings (not shown) may also be formed in needle (20), and may be in fluid communication with second lumen (26). For instance, such external openings may be configured in accordance with the teachings of U.S. Pub. No. 2007/0032742, entitled “Biopsy Device with Vacuum Assisted Bleeding Control,” published Feb. 8, 2007, the disclosure of which is incorporated by reference herein. Cutter (50) may also include one or more side openings (not shown). Of course, as with other components described herein, such external openings in needle (20) and cutter (50) are merely optional.

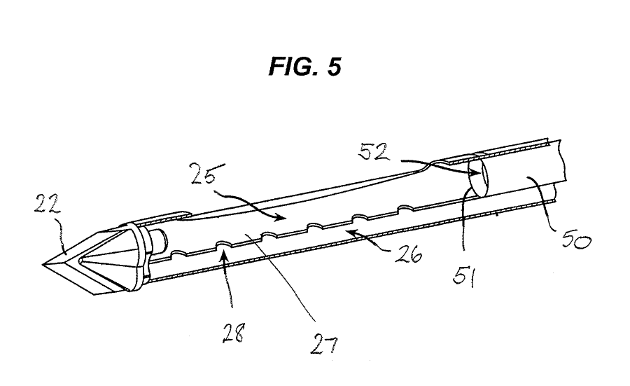

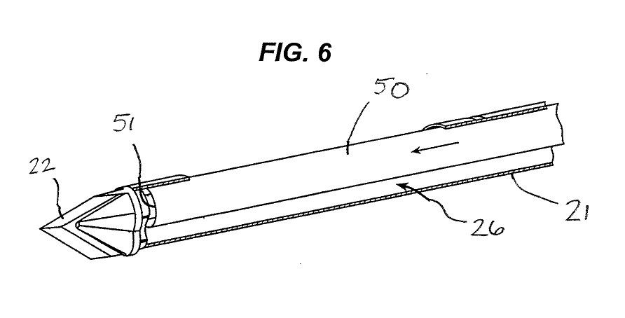

FIG. 5 depicts cutter (50) fully retracted by cutter actuator (110), such that lateral aperture (23) is completely unobstructed by cutter (50). In this configuration tissue can prolapse through lateral aperture (23) within first lumen (25) under the force of gravity, due to internal pressure of the tissue (e.g., caused by displacement of the tissue upon insertion of needle (20), etc.), and/or with vacuum provided through second lumen (26) and transmitted through openings (28) and/or by vacuum provided through cutter lumen (52). FIG. 6 depicts cutter (50) after it has been advanced to close off lateral aperture (23) once tissue has been captured within first lumen (25). With the tissue severed, it is captured within cutter lumen (52) and ready for proximal transport to tissue sample holder (40). Such proximal transport of tissue through cutter lumen (52) to reach tissue sample holder (40) may be provided by drawing a vacuum through the proximal portion of cutter lumen (52) (e.g., behind the captured tissue sample) while venting a distal portion of cutter lumen (52) (e.g., in front of the captured tissue sample) to provide a pressure differential. Alternatively, tissue samples severed by cutter (50) may be communicated proximally to tissue sample holder (40) or be otherwise dealt with in any other suitable fashion.

While the above paragraphs provide an enabling description of an exemplary biopsy device (10) and its use, further description as well as exemplary methods of operation are provided with the teachings of U.S. Non-Provisional patent application Ser. No. 12/709,624, entitled “Spring Loaded Biopsy Device,” filed Feb. 22, 2010, and U.S. Pub. No. 2008/0214955, entitled “Presentation of Biopsy Sample by Biopsy Device,” published Sep. 4, 2008, the disclosures of which are incorporated by reference herein. Of course, the above examples of construction and use of biopsy device (10) are merely illustrative. Other suitable ways in which biopsy device (10) may be made and used will be apparent to those of ordinary skill in the art in view of the teachings herein.

II. Exemplary Needle Tip Protectors

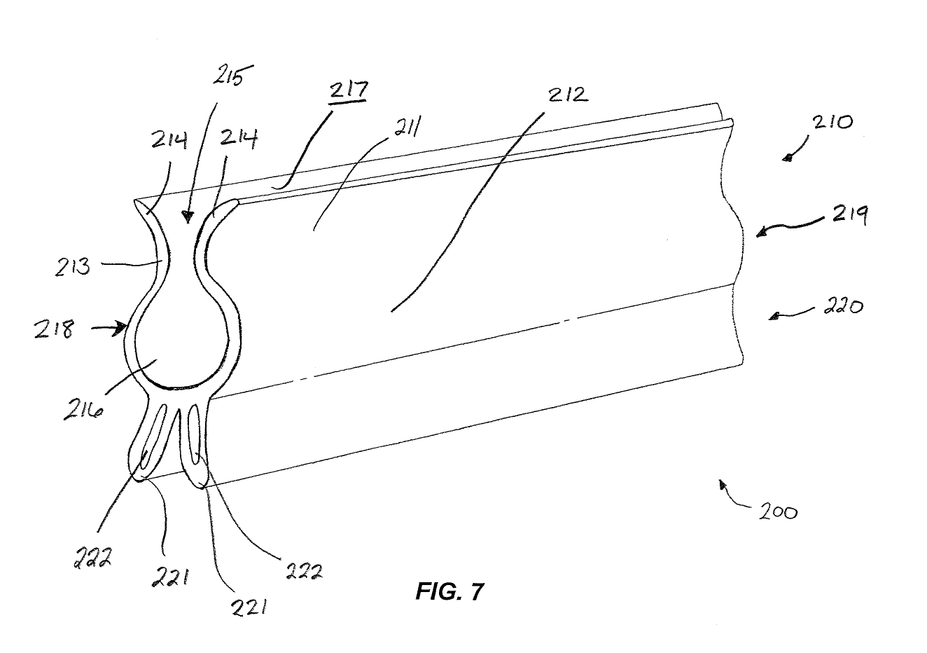

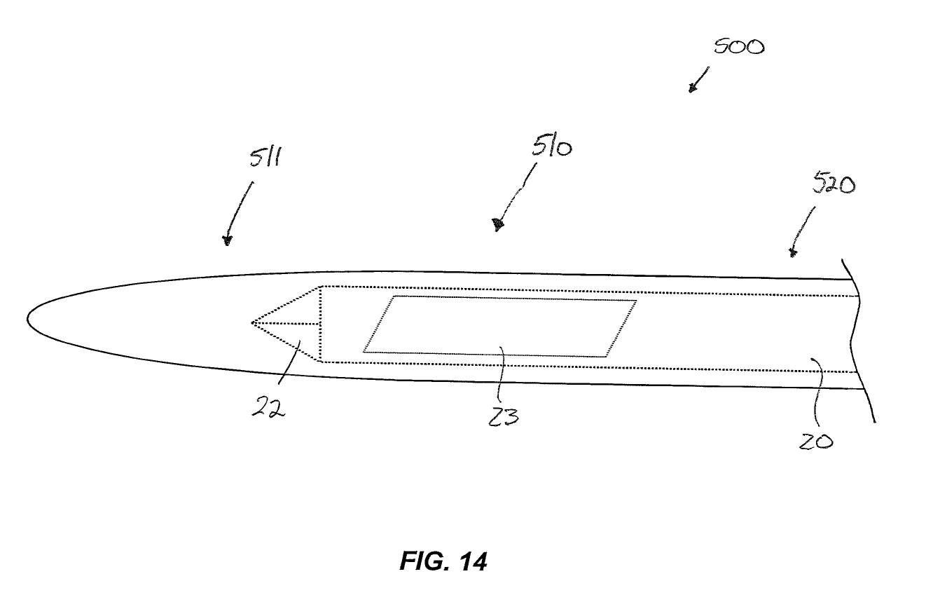

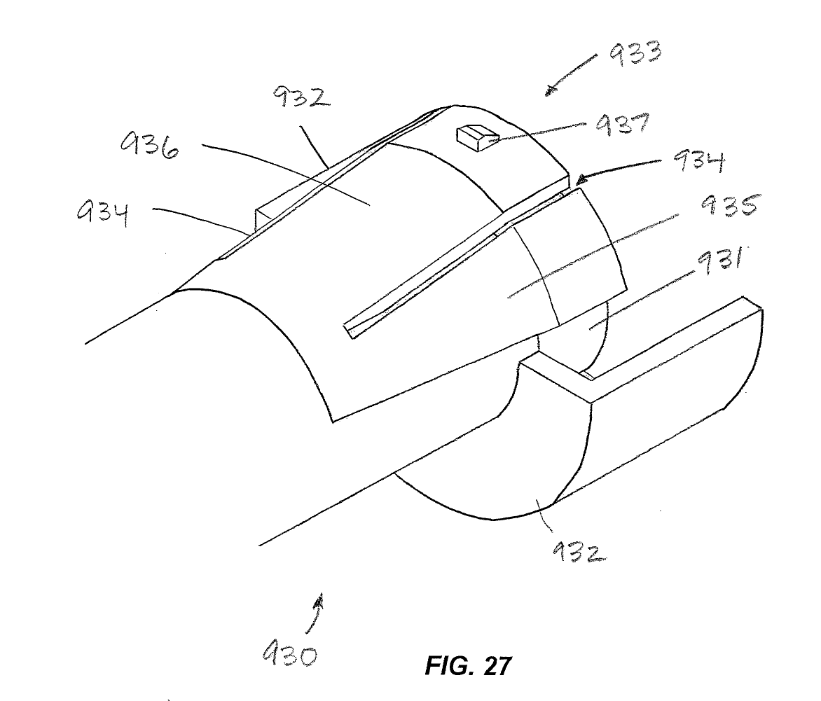

Another component that may be used with a biopsy device, such as biopsy device (10) described above, is a needle tip protector. Generally, a needle tip protector may protect all or a portion of a needle cannula (21) and tissue piercing tip (22) from damage during handling. A needle tip protector may also protect those exposed to biopsy device (10) from injury by unintentional contact with the sharp tissue piercing tip (22). Several exemplary needle tip protector versions for use with an exemplary biopsy device are presented in the following sections. FIGS. 7-37 depict views of some of the exemplary needle tip protectors described below. Of course, other suitable alternative versions, features, components, configurations, and functionalities for a needle tip protector will be apparent to those of ordinary skill in the art in view of the teachings herein.

A. Exemplary Detachable, Laterally and Proximally Applied, Needle Tip Protectors

Some needle tip protectors may be installed and completely removed from a biopsy device (10) in such a fashion where a user's hand is not required to be positioned distal of the sharp needle tip (22). Such an approach may reduce the risk that a user will accidently contact needle tip (22) when installing or removing the needle tip protector. Several examples of such needle tip protectors are described in the paragraphs that follow, while other examples will be apparent to those of ordinary skill in the art in view of the teachings herein.

1. Single Piece Extruded Elastomeric

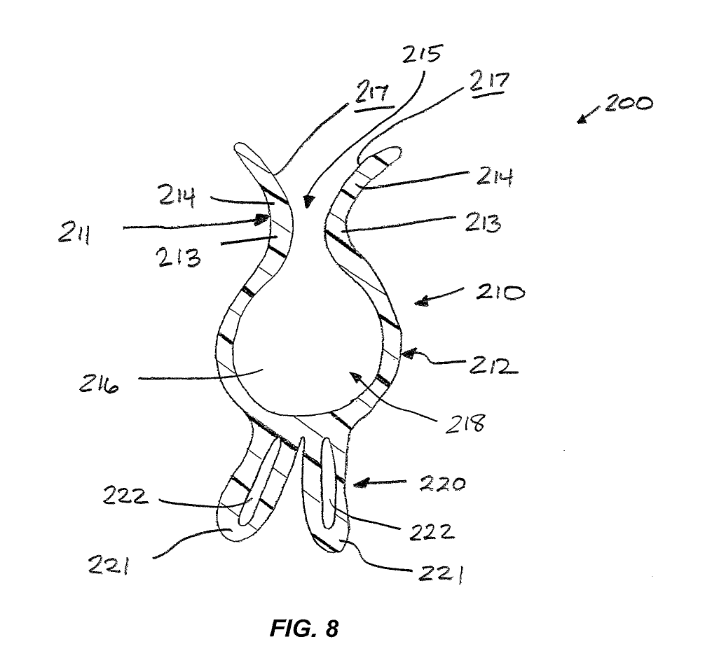

FIGS. 7-8 show an exemplary needle tip protector (200). In the present example, needle tip protector (200) comprises a tube (210) and grippers (220). Tube (210) comprises an upper region (211) and lower region (212). Upper region (211) includes a collar (213), a pair of outwardly extending shelves (214), and an opening (215) extending along the length of tube (210). Collar (213) is presented as a narrowing portion of tube (210) along upper region (211). Collar (213) is configured to receive cannula (21) of needle (20). In the present example, collar (213) has a width that is less than that of the diameter of needle (20). However, needle tip protector (200) is constructed of an elastomeric material such that collar (213) has resilient properties that allow it to flex and compress to permit the cannula (21) of needle (20) to pass by collar (213) and come to rest within the larger hollow region (216) of tube (210). Shelves (214) provide surfaces (217) for supporting cannula (21) of needle (20) before needle tip protector (200) is fully installed on needle (20). Surfaces (217) help maintain needle (20) in the proper position for subsequent placement within hollow region (216) of tube (210). For instance, in the present example, shelves (214) reduce side-to-side movement of needle (20) during application of needle tip protector (200).

Lower region (212) connects with grippers (220). Grippers (220) have an inverted “V” shape with outermost portions (221) positioned furthest from one another and furthest from tube (210). In the illustrated version, grippers (220) contain hollow portions (222) that extend along the length of grippers (220). Of course, grippers (220) need not necessarily contain hollow portions (222) in all versions.

In the present example, needle tip protector (200) is extruded as a single piece of elastomeric material that is cut to length. Thus, tube (210) and grippers (220) are unitary. As mentioned, the elastomeric construction of needle tip protector (200) provides resilient properties to needle tip protector (200) such that collar (213) flexes and compresses to allow cannula (21) of needle (20) to pass through opening (215) and into hollow region (216) of tube (210). In the illustrated version, grippers (220) are operably configured such that when grippers (220) are grasped and pressed together, outward forces are transferred to shelves (214) and collar (213) thereby expanding opening (215) to permit cannula (21) of needle (20) to more easily be positioned within hollow region (216) of tube (210). The degree of resiliency for needle tip protector (200) may be altered, as well as the configuration of grippers (220), to assist with expanding opening (215) when grippers (220) are grasped and pressed together and needle (20) is installed or removed from needle tip protector (200). In some versions, grippers (220) may be omitted entirely. Also, materials other than elastomeric materials may be adapted for use as needle tip protector (200), and such other materials will be apparent to those of ordinary skill in the art in view of the teachings herein. For instance, while in the present example, use of elastomeric materials permits components of tube (210) to both flex and compress, in some other versions materials of construction may permit flexibility of components with minimal or no compression, yet still provide acceptable function as a needle tip protector.

In use, needle tip protector (200) can be applied to needle (20) from any position lateral to needle (20). For instance, needle tip protector (200) may be applied to needle (20) from above, below, or from either side of needle (20). To apply needle tip protector (200) to needle (20), shelves (214) are located along the side of cannula (21) of needle (20) such that needle (20) is aligned with opening (215) and above collar (213). Grippers (220) are then squeezed together while pushing needle tip protector (200) against the side of cannula (21) of needle (20) causing shelves (214) to flex to a more open position while portions of needle tip protector (200) contacting cannula (21) of needle (20) compress. This action permits needle tip protector (200) to laterally slide over cannula (21) of needle (20) in a direction that is transverse to the longitudinal axis of cannula (21). Once needle (20) is within hollow region (216) of tube (210), grippers (220) are released and shelves (214) and collar (213) return to their initial positions and forms. In some versions, a benefit of applying needle tip protector (200) in this fashion may be that a user's hand is not required to be positioned distal of needle tip (22) to apply needle tip protector (200). Also, the movement involved in installing needle tip protector (200) is perpendicular to needle tip (22). These features of needle tip protector (200) may reduce the risk of accidental contact with needle tip (22) when installing needle tip protector (200). Of course, in some other versions needle tip protector (200) may be applied to needle (20) by aligning needle tip (22) with an open proximal end (218) of tube (210) and sliding needle tip protector (200) axially over needle (20), in a direction that is parallel to the axis defined by cannula (21).

To remove needle tip protector (200) from needle (20), grippers (220) are squeezed and pulled laterally in a direction away from cannula (21) of needle (20). This action again causes shelves (214) to flex outward, thereby making opening (215) larger to permit cannula (21) to emerge from hollow region (216) of tube (210). Also during removal of needle tip protector (200), the resilient properties of collar (213) permit collar (213), and/or shelves (214) for that matter, to compress when contacting cannula (21), thereby aiding in removal of needle (20) from within hollow region (216) of tube (210). As with application of needle tip protector (200), a benefit of removing needle tip protector (200) as described above may be that a user's hand is not required to be positioned distal of needle tip (22) to remove needle tip protector (200). Also, the movement involved in removing needle tip protector (200) is perpendicular to needle tip (22). These features of needle tip protector (200) may reduce the risk of accidental contact with needle tip (22) when removing needle tip protector (200). Of course, in some other versions, needle tip protector (200) may be removed from needle (20) by sliding needle tip protector (200) axially off needle (20), with needle (20) exiting open proximal end (218) of tube (210).

In the present example, needle tip protector (200) has an open distal end (219). In some other versions, distal end (219) may be closed. Needle tip protector (200) of the present example is configured to cover a substantial portion of needle (20), though it should be understood that needle tip protector (200) may be configured to cover more or less of needle (20). For example, in some versions needle tip protector (200) may cover substantially all of needle (20); while in some other versions needle tip protector (200) may cover only a distal portion of needle (20). Needle tip protector (200) may be configured for use across 8 gauge and 10 gauge needles, among others, and for all lengths, e.g., 9 centimeters, 12 centimeters, 15 centimeters, among others. Other suitable alternative versions, features, components, configurations, and functionalities of needle tip protector (200) will be apparent to those of ordinary skill in the art in view of the teachings herein.

2. Living Hinge Trap

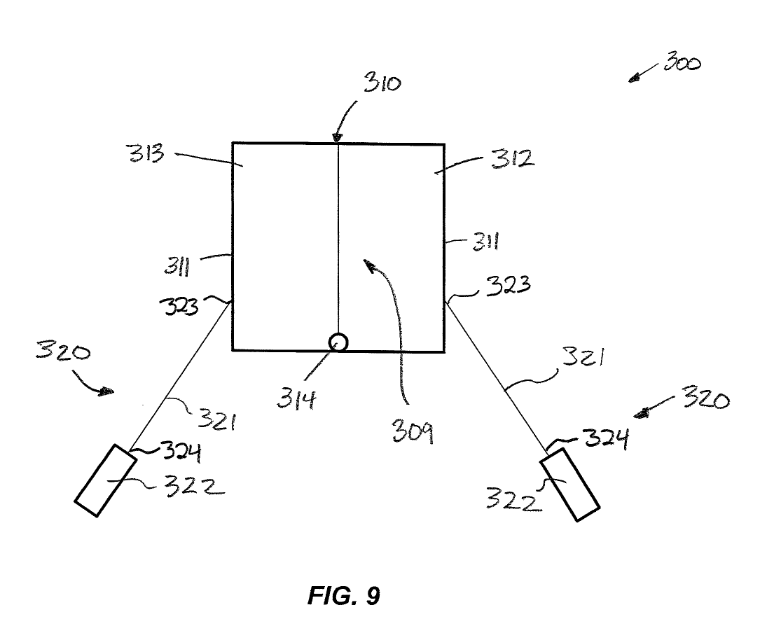

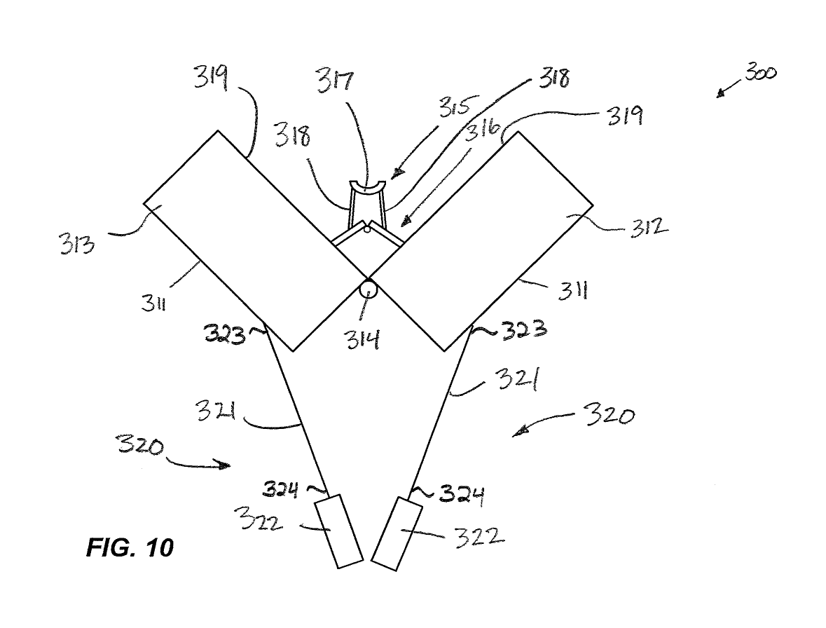

FIGS. 9-10 show another exemplary needle tip protector (300). Needle tip protector (300) of this example comprises housing (310) and grippers (320). Grippers (320) comprise legs (321) and boots (322). Legs (321) are connected to outer sides (311) of housing (310) at a respective first ends (323). Boots (322) are connected to legs (321) at respective second ends (324). Housing (310) comprises a first half (312) and a second half (313). First half (312) and second half (313) are pivotally connected about a pivoting member (314). A shelf (315) and a living hinge (316) are located within housing (310). Shelf (315) is configured to hold cannula (21) of needle (20), and shelf (315) comprises a support section (317) and a stand section (318). Living hinge (316) is connected with stand section (318) of shelf (315) as shown in the illustrated version. Living hinge (316) is further connected with inner sides (319) of housing (310).

FIG. 9 shows needle tip protector (300) in a closed position. Needle tip protector (300) is moved to the open position shown in FIG. 10 by pressing grippers (320) toward each other. With grippers (320) connected to outer sides (311) of housing (310), as grippers (320) are moved toward each other, first half (312) and second half (313) of housing (310) pivot about pivoting member (314) thus opening housing (310) to reveal shelf (315) and living hinge (316). In the present example, as housing (310) opens, living hinge (316) also assumes a new orientation, moving from a regular “V” configuration when housing (310) is closed as in FIG. 9, to an inverted “V” configuration when housing (310) is opened as in FIG. 10. In its inverted “V” configuration, living hinge (316) biases housing (310) in the open position such that needle tip protector (300) remains open once grippers (320) have been sufficiently moved toward one another without the need for continuously pressing grippers (320) toward each other.

To protect needle tip (22) with needle tip protector (300), needle (20) of biopsy device (10) is first positioned on support section (317). Downward force is then applied to needle (20), which causes shelf (315) to push against living hinge (316). The action of shelf (315) pushing against living hinge (316) causes living hinge (316) to return to its closed configuration, e.g., in the regular “V” configuration. This removes the biasing force that the living hinge (316) exerted on housing (310) to maintain housing (310) in the open position, thereby causing housing (310) to close around needle (20).

In the present example, housing (310) of needle tip protector (300) includes a closed distal end (309) and a hollow interior. An open proximal end (not shown) is opposite closed distal end (309). With this configuration, needle tip (22), along with a portion of cannula (21) of needle (20), is covered by needle tip protector (300) while the remaining portions of needle (20) and biopsy device (10) extend proximally from needle tip protector (300). Based on the teachings herein, it will be apparent to those of ordinary skill in the art that the size and proportions of needle tip protector (300) may be altered to provide protection for more or less of needle (20), or even provide protection for other components of biopsy device (10), e.g., needle hub (24), housing (31), etc.

In use, needle tip protector (300) may be used as a standalone component, or needle tip protector (300) may be used in conjunction with other components, e.g., a blister pack or tray. Needle tip protector (300) may be used for pre-procedure protection of needle (20), post-procedure protection of needle (20), or both. In the present example, needle tip protector (300) is used for safely recapping needle (20) after needle (20) has been used in a procedure. To this end, in the present example, once needle tip protector (300) is opened to the position shown in FIG. 10, needle tip protector (300) is set back into a blister pack or tray within which the device was originally shipped. After the procedure is complete, a user places needle (20) of biopsy device (10) on support section (317) of shelf (315) and presses needle (20) downward. Living hinge (316) responds to the downward force by returning to its inverted “V” configuration. This action causes first half (312) and second half (313) of housing (310) to close around needle (20). Other suitable alternative versions, features, components, configurations, and functionalities of needle tip protector (300) will be apparent to those of ordinary skill in the art in view of the teachings herein.

3. Living Hinge Extending Arms

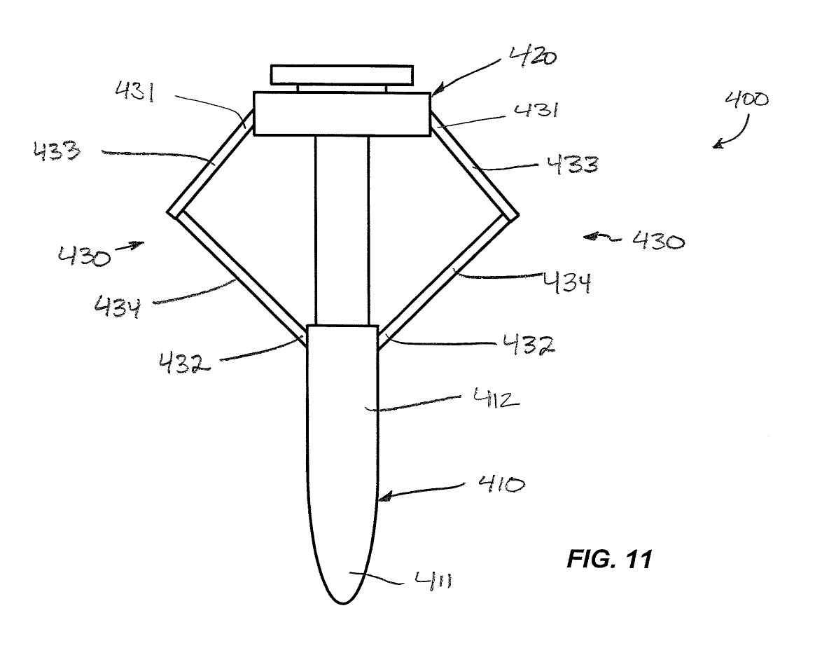

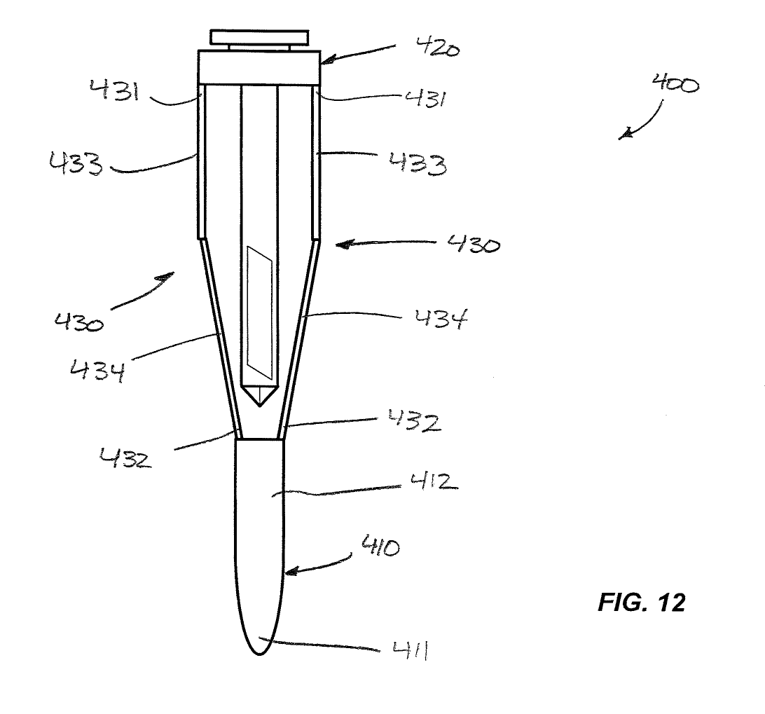

FIGS. 11-12 show another exemplary needle tip protector (400). Needle tip protector (400) of this example comprises cover (410), locking member (420), and a pair of living hinges (430). Living hinges (430) comprise proximal ends (431) and distal ends (432). Proximal ends (431) pivotally connect to each side of locking member (420). Distal ends (432) pivotally connect to each side of cover (410). In the present example, cover (410) comprises a cap-style cover having a blunt or rounded tip (411) that completely surrounds needle tip (22). Cover (410) also comprises a body portion (412) that extends proximally to cover lateral aperture (23). In some versions, cover (410) is constructed from a rigid plastic material that is sufficiently strong to prevent needle tip (22) from penetrating cover (410). In some versions, cover (410) includes a cushioned portion within tip (411) such that needle tip (22) is protected from damage should it contact the interior of cover (410). In the present example, cover (410) surrounds needle tip (22) and lateral aperture (23) without needle tip (22) contacting the interior of tip (411) of cover (410). Thus, cover (410) may be considered as “hovering” around needle tip (22) and lateral aperture (23). This configuration for cover (410) is achieved by the configuration of living hinges (430). Specifically, living hinges (430) are configured with a length and spring bias that holds cover (410) in this hovering fashion without pulling cover (410) so far proximally that it contacts needle tip (22). Of course, based on the teachings herein, other positions for cover (410), including positions where cover (410) contacts needle tip (22), will be apparent to those of ordinary skill in the art.

In the present example, locking member (420) is configured to snap-fit engage needle hub (24), thereby securing needle tip protector (400) to biopsy device (10). In some versions, locking member (420) is configured to snap-fit engage needle (20) itself instead of, or in addition to, snap-fit engaging needle hub (24). To achieve a snap-fit engagement between locking member (420) and needle hub (24), any suitable snap-fit engagement means may be used. For example, in some versions, the underside of locking member (420) comprises two resilient locking arms (not shown) that extend downward and engage needle hub (24) when locking member (420) is pressed proximally against needle hub (24) with sufficient force. In some such versions, the two locking arms pivotally connect with the sides of locking member (420) and include upper portions that, when depressed, pivot the two locking arms outward away from needle hub (24) to disengage the locking arms from needle hub (24). Other suitable structures to achieve a snap-fit connection between locking member (420) and needle hub (24) and/or needle (20) will be apparent to those of ordinary skill in the art in view of the teachings herein.

In the present example, living hinges (430) comprise respective first sections (433) and second sections (434) that are joined at approximately a forty-five degree orientation as shown in FIG. 11. However, other orientations for living hinges (430) will be apparent to those of ordinary skill in the art in view of the teachings herein. When living hinges (430) have the orientation shown in the present example, living hinges (430) are in a neutral position, e.g., not under tension. In other words, living hinges (430) are resiliently biased to maintain the positions shown in FIG. 11. Thus, living hinges (430) will remain in this position, holding cover (410) in the same relative position. By matching the length of needle (20) with the appropriate size of needle tip protector (400), cover (410) will remain surrounding needle tip (22) and lateral aperture (23) when needle tip protector (400) is installed on needle (20) and living hinges (430) are in the neutral position as shown in FIG. 11.

Referring now to FIG. 12, a user has applied lateral force to living hinges (430) along first sections (433) and/or at the transition point between first and second sections (433, 434). This action results in living hinges (430) extending distally toward needle tip (22). This extension drives cover (410) distally, revealing needle tip (22) and lateral aperture (23). When living hinges (430) are fully extended distally, cover (410) assumes a position distal of needle tip (22) thereby permitting needle tip protector (400) to be removed from biopsy device (10) by disengaging snap-fit locking member (420) from needle hub (24) and/or needle (20). To reinstall needle tip protector (400), a user would actuate living hinges (430) by applying inward lateral force as described above, align cover (410) with needle tip (22), snap-fit locking member (420) to needle hub (24) and/or needle (20), then release living hinges (430) to remove the lateral force. In response to removal of the lateral force, living hinges (430) return to their neutral position shown in FIG. 11, and cover (410) is drawn proximally, thereby covering needle tip (22) and lateral aperture (23). With the above described configuration, needle tip protector (400) may be applied and removed from biopsy device (10) without the user's hand ever needing to be distal of needle tip (22). As mentioned previously, such an application and removal technique may aid in reducing the risk of accidental contact with sharp needle tip (22) during covering or uncovering needle tip (22) with needle tip protector (400).

In some versions, cover (410) may be fitted with a seal (not shown) around its proximal end. Adding a seal in this fashion may provide that lateral aperture (23) may be sealed within cover (410) when needle tip protector (400) is installed. In this example and in various other examples described herein, such a sealed configuration may permit needle (20) to be primed with saline or some other treatment fluid prior to performing a biopsy procedure. Such priming of needle (20) may lubricate needle (20), which may reduce the force required for insertion of needle (20) in tissue. Such priming of needle (20) may also lubricate the inside of cutter (50), which may reduce the likelihood of a “dry tap” during a subsequent biopsy procedure by facilitating proximal transport of tissue through cutter lumen (52). As will be apparent to those of ordinary skill in the art, such sealing and priming may be provided in various other examples and versions of needle tip protectors described herein. In the present example, needle tip protector (400) is configured for use both before and after a procedure. Other suitable alternative versions, features, components, configurations, and functionalities of needle tip protector (400) will be apparent to those of ordinary skill in the art in view of the teachings herein.

4. Hook and Lock

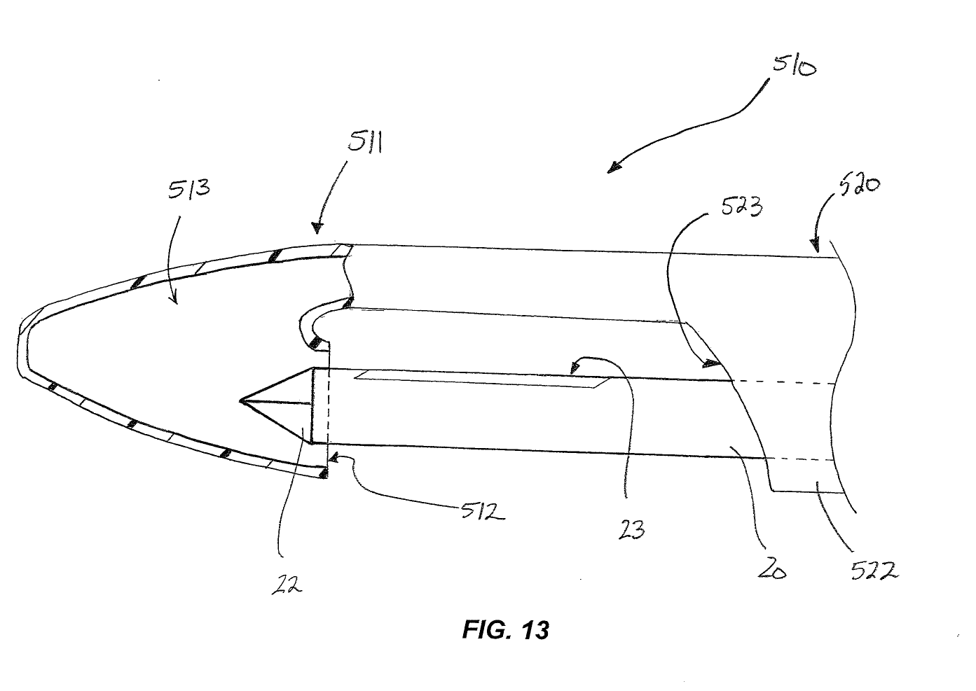

FIGS. 13-15 show another exemplary needle tip protector (500). Needle tip protector (500) of this example comprises a hook member (510) and a locking member (520). In the present example, hook member (510) comprises a tip (511) and a proximally presented inlet (512). Tip (511) has an interior void space (513) communicating with inlet (512). Interior void space (513) is configured to house needle tip (22). Tip (511) further has a blunt shape with a closed distal end. Inlet (512) is configured to receive needle tip (22) and permit needle tip (22) to be positioned within interior void space (513), thereby preventing exposure of needle tip (22) to a user and protecting needle tip (22) during handling of biopsy device (10). In some versions, tip (511) is configured to house more than just needle tip (22). For instance, in some versions tip (511) is configured to house both needle tip (22) and enough of needle (20) to position lateral aperture (23) within interior void space (513). In some such versions, inlet (512) and tip (511) may be configured with sealing structures such that lateral aperture (23) is sealed within interior void space (513) so that needle (20) may be primed with saline or some other fluid. In some versions, tip (511) may be configured with cushioning material (not shown) within interior hollow space (513) for protecting needle tip (22) from damage if over-inserted through inlet (512). Based on the teachings herein, other configurations for hook member (510) having tip (511) and inlet (512) will be apparent to those of ordinary skill in the art.

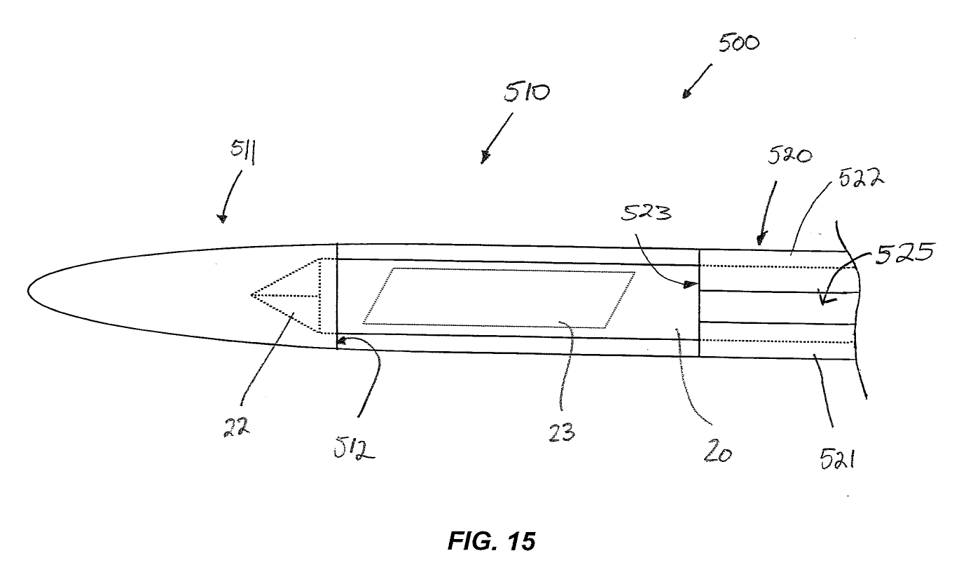

Locking member (520) comprises a first locking portion (521), a second locking portion (522), and a hollow interior (523) defined by first and second locking portions (521, 522). First and second locking portions (521, 522) are separated by a gap (525), as seen in FIG. 15, and are operatively configured to snap-fit around needle (20) thereby securing needle tip protector (500) to needle (20). Of course in some versions locking member (520) may instead, or in addition, snap-fit around needle hub (24) or another structure of biopsy device (10). In the present example, first and second locking portions (521, 522) are constructed of a plastic that has resilient properties such that first and second locking portions (521, 522) flex outward under force when contacting the outer diameter of needle (20). Once needle (20) is positioned within hollow interior (523) of locking member (520) first and second locking portions (521, 522) return to their un-flexed state, providing a snugly secure fit around needle (20). In such versions, the snap-fit of locking member (520) with needle (20) keeps needle tip protector (500) securely mounted to needle (20). Various other structures for securing locking member (520) to needle (20) or some other structure will be apparent to those of ordinary skill in the art in view of the teachings herein.

In an exemplary use, a user holds needle tip protector (500) near a proximal portion, e.g., near locking member (520). The user then hooks needle tip (22) by aligning inlet (512) with needle tip (22) and moving needle tip protector (500) proximally. This action causes needle tip (22) to be positioned within interior void space (513). With needle tip (22) safely within tip (511), locking member (520) is pushed downward against the outer diameter of needle (20) such that first and second portions (521, 522) of locking member (520) snap-fit around needle (20). To remove needle tip protector (500), a user disengages locking member (520) from needle (20) by lifting locking member (520) upwards. This upwards lifting action causes first and second portions (521, 522) to contact the outer diameter of needle (20) and resiliently move outward from needle (20), thereby permitting needle (20) to be repositioned outside of hollow space (523). The user may then grasp needle tip protector (500) along a proximal portion, e.g., the locking member (520), and advance needle tip protector (500) in a distal direction until needle tip (22) is removed from interior void space (513). At this point, needle tip protector (500) may be removed entirely from needle (20) and biopsy device (10). With needle tip protector (500) having at least some of the features described above, it will be apparent to those of ordinary skill in the art that a user may install and remove needle tip protector (500) without ever having their hands positioned distal of needle tip (22), thereby preventing inadvertent contact with needle tip (22). Other suitable alternative versions, features, components, configurations, and functionalities of needle tip protector (500) will be apparent to those of ordinary skill in the art in view of the teachings herein.

5. Proximal Upper Shield

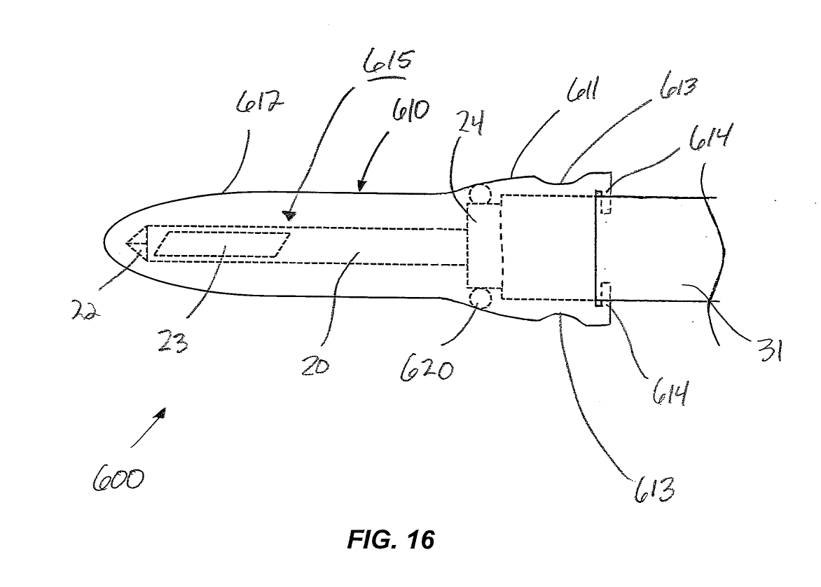

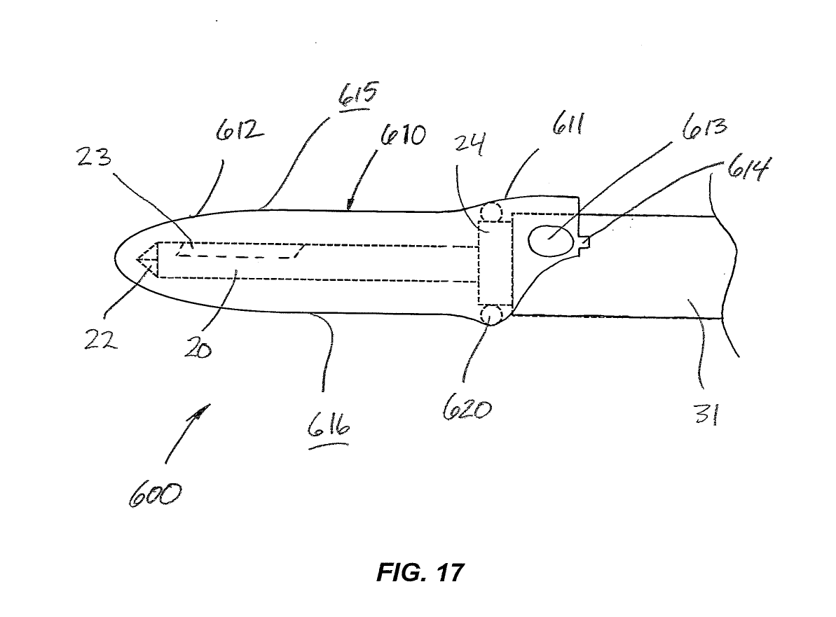

FIGS. 16-17 show another exemplary needle tip protector (600). Needle tip protector (600) of this example comprises a body (610), which is substantially hollow and has a proximal portion (611) and a distal portion (612). Distal portion (612) has a closed end while proximal portion (611) has an open end. Proximal portion (611) comprises dual finger holds (613) as well as dual side latches (614). Side latches (614) are in communication with finger holds (613), such that side latches (614) resiliently flex outward from a central longitudinal axis of needle tip protector (600) as finger holds (613) are pressed. Side latches (614) are configured to engage corresponding recesses (not shown) in housing (31) of biopsy device (10). When side latches (614) are engaged with the recesses in housing (31), needle tip protector (600) is securely yet removably attached to biopsy device (10); and distal portion (612) of body (600) surrounds needle (20) of biopsy device (10). As best shown in FIG. 17, body (610) can be considered to have a top surface (615) and a bottom surface (616). In the present example, top surface (615) extends further proximally than bottom surface (616). This configuration for body (610) provides proximal portion (611) with decreasing coverage of housing (31) of biopsy device (10) as proximal portion (611) extends proximally. Thus, the cross sectional area of proximal portion (611) decreases as proximal portion (611) extends proximally.

With the above configuration for proximal portion (611), a user can remove needle tip protector (600) from biopsy device (10) without extending the user's hand distal of needle tip (22). More specifically, to remove needle tip protector (600), a user may grasp proximal portion (611) and press finger holds (613). This action causes side latches (614) to resiliently flex outward from housing (31) such that side latches (614) disengage the recesses (not shown) in housing (31). An inwardly protruding fulcrum feature (not shown) may be provided in needle tip protector (600), between each finger hold (613) and its corresponding latch (614) to provide such outward flexing of latches (614) in response to inward pressing on finger holds (613). With latches (614) sufficiently cleared from housing (31), the user then advances needle tip protector (600) distally until needle tip (22) clears bottom surface (616) of body (610). With needle tip (22) clear of bottom surface (616) of body (610), top surface (615) of body (610) still extends far enough proximally to cover needle tip (22). However, because needle tip (22) is clear of bottom surface (616), needle tip protector (600) can be removed by raising needle tip protector (600) upward instead of the user needing to advance needle tip protector (600) further distally. Thus this arrangement allows the user to remove—and in the reverse procedure, install—needle tip protector (600) without placing the user's hand distal of needle tip (22).

In the present example, needle tip protector (600) further comprises seal (620). Seal (620) is positioned within the hollow interior of body (610). Seal (620) is further positioned such that when needle tip protector (600) is installed on biopsy device (10), seal (620) abuts needle hub (24) thereby fluidly sealing needle (20) within the hollow distal portion (612) of body (610). With needle (20) sealed within distal portion (612), needle (20) can be primed with a treatment fluid before a procedure, e.g., saline or other fluids can be delivered to lateral aperture (23) without leaking. It should be understood that seal (620) may be omitted entirely; or be repositioned to create a seal by abutting components of biopsy device (10) other than needle hub (24). In the present example, seal (620) is shown as an o-ring. Of course other configurations for seal (620) will be apparent to those of ordinary skill in the art in view of the teachings herein. Still yet other suitable alternative versions, features, components, configurations, and functionalities of needle tip protector (600) will be apparent to those of ordinary skill in the art in view of the teachings herein.

B. Exemplary Detachable, Distally Applied Needle Tip Protectors

Some needle tip protectors may be installed and removed by placing the needle tip protector distal to the needle tip (22) and advancing the needle tip protector proximally to cover the needle tip (22). Such needle tip protectors can protect the needle tip (22) from damage during handling, and can reduce the risk that the user will accidently contact the sharp needle tip (22) when handling or nearby the biopsy device. Several examples of such needle tip protectors are described in the paragraphs that follow, while other examples will be apparent to those of ordinary skill in the art in view of the teachings herein.

1. Elastomeric Seal

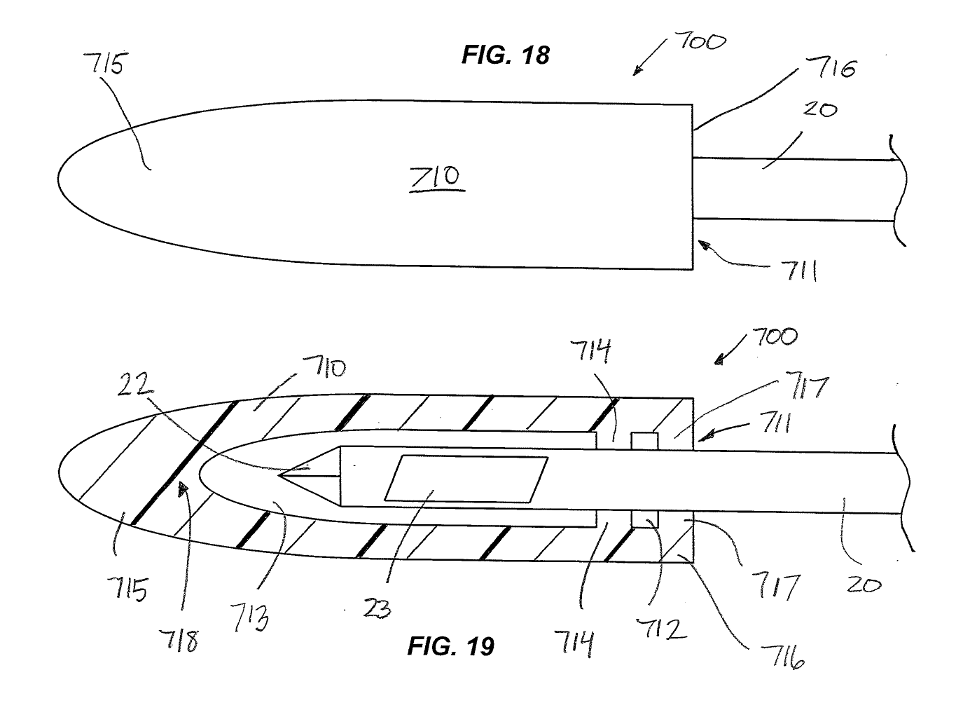

FIGS. 18-19 show another exemplary needle tip protector (700). Needle tip protector (700) of this example comprises a one-piece elastomeric body (710) that defines a hollow interior (711). Hollow interior (711) is divided into a proximal region (712) and a distal region (713) by a first inwardly protruding annular rib (714). In some versions, first rib (714) comprises a plurality of ribs. Such a plurality of ribs may be located at a common longitudinal position and may extend only part of the circumference of the inner diameter of hollow interior (711). In addition or in the alternative, a plurality of ribs may be located at different longitudinal positions. Body (710) comprises a closed distal end (715) and an open proximal end (716). Open proximal end (716) includes a second inwardly protruding annular rib (717). In some versions, second rib (717) comprises a plurality of ribs. Such a plurality of ribs may be located at a common longitudinal position and may extend only part of the circumference of the inner diameter of hollow interior (711). In addition or in the alternative, a plurality of ribs may be located at different longitudinal positions. In the present example, second rib (717) defines the opening in open proximal end (716).

Distal end (715) comprises a cushion (718) that provides material that will not damage needle tip (22) if needle tip (22) is advanced distally enough to contact cushion (718), e.g., if needle (20) is dropped and lands on distal end (715). In the present example, cushion (718) is comprised of the same elastomeric material used for the entire needle tip protector (700). Of course, in some other versions cushion (718) may be comprised of different elastomeric materials or other materials altogether. Also, in the present example ribs (714, 717) are comprised of the same elastomeric material used for the entire needle tip protector (700). Of course in some other versions, ribs (714, 717) may be comprised of different elastomeric materials or other materials altogether.

When used in conjunction with needle (20), needle tip protector (700) is installed by sliding needle tip protector (700) over needle tip (22) and lateral aperture (23) of needle (20) such that ribs (714, 717) are positioned proximal of lateral aperture (23). Ribs (714, 717) have resilient properties such that ribs (714, 717) compress against the outer surface of needle (20) during installation of needle tip protector (700), thereby frictionally retaining needle tip protector (700) securely to needle (20) while also creating a dual-barrier seal with the outer surface of needle (20). With the secure and sealed connection between needle tip protector (700) and needle (20) being proximal from lateral aperture (23), needle (20) may be primed before a procedure with saline or some other treatment fluid. With the resilient nature of ribs (714, 717), needle tip protector (700) can be used with any of several gauge needles. Of course the size and geometry of ribs (714, 717) may be altered to accommodate even more variations in needles.

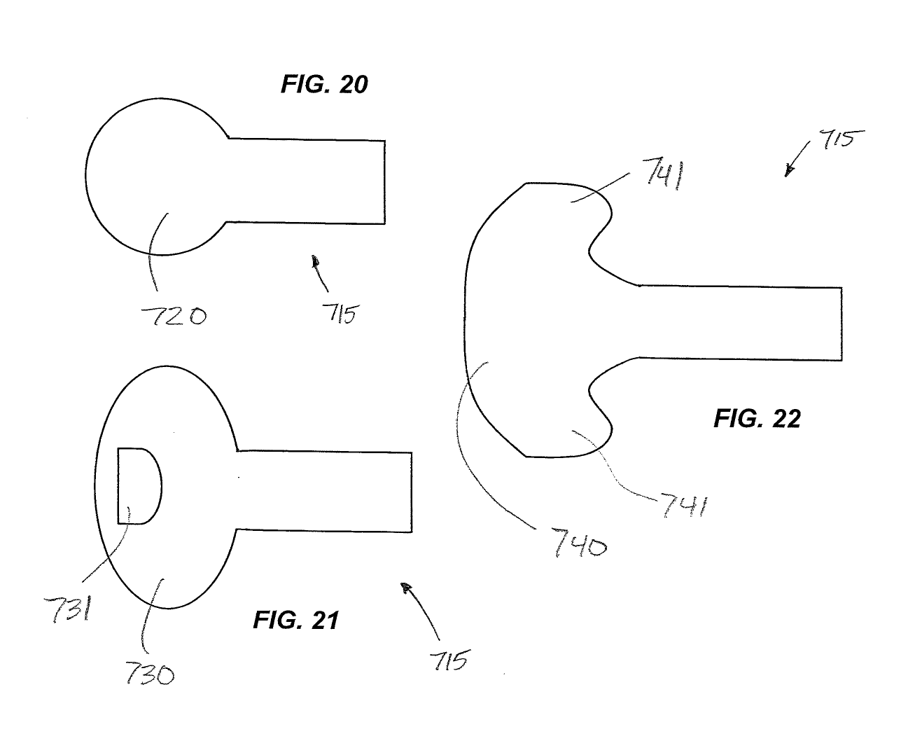

FIGS. 20-22 show other exemplary geometric variations to distal end (715) of needle tip protector (700). These variations provide additional gripping areas to assist a user removing needle tip protector (700) from biopsy device (10). As shown in FIG. 20, one variation to distal end (715) involves use of a solid ball (720) at distal end (715). As shown in FIG. 21, another variation involves use of an enlarged distal end (730) incorporating a finger tab (731). As shown in FIG. 22, another variation involves use of an enlarged distal end (740) incorporating dual finger hooks (741). Based on the teachings herein, other variations to distal end (715) that may aid in a user's efforts to remove needle tip protector (700) will be apparent to those of ordinary skill in the art. Still yet other suitable alternative versions, features, components, configurations, and functionalities of needle tip protector (700) will be apparent to those of ordinary skill in the art in view of the teachings herein.

2. Hinged Seal

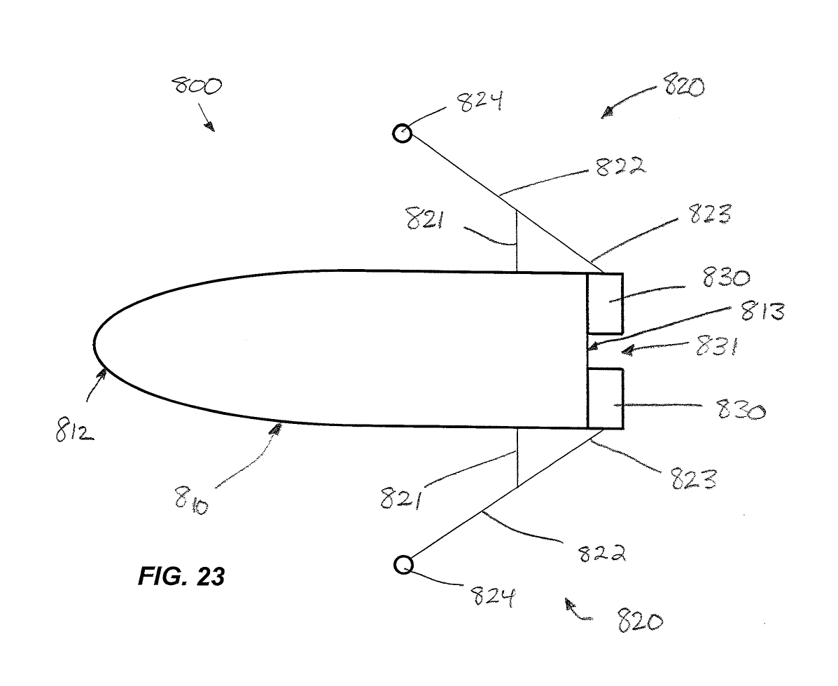

FIGS. 23-25 show another exemplary needle tip protector (800). Needle tip protector (800) of this example comprises a body (810), living hinges (820), and elastomeric boots (830). In the present example, body (810) comprises a polycarbonate structure having a hollow interior (811) for receiving needle tip (22) and lateral aperture (23) of needle (20). Body (810) further comprises a closed distal end (812) and an open proximal end (813).

Living hinges (820) comprise joining members (821) and pivoting arms (822). Joining members (821) connect pivoting arms (822) with body (810). In the present example, joining members (821) connect with pivoting arms (822) at about the midpoint of pivoting arms (822), though it should be understood that joining members (821) may be coupled with pivoting arms (822) at any other suitable location along the length of pivoting arms (822) (e.g., to provide a desired amount of leverage, etc.). Pivoting arms (822) have proximal ends (823) that connect with elastomeric boots (830). Handlebars (824) are positioned at the opposing end of pivoting arms (822).

Boots (830) are comprised of an elastomeric material with sufficient resilient properties that allow boots (830) to compress upon subjecting boots (830) to a force. When a force applied to boots (830) is removed, boots (830) return to their natural uncompressed form. In the present example, boots (830) have a semi-circular shape that mirrors approximately half of the cross sectional shape of body (810). However, boots (830) of the present example are configured such that there is a space (831) between boots (830) when boots (830) are secured against needle (20). Of course, in some versions space (831) is omitted entirely and boots (830) contact each other. Based on the teachings herein, other configurations for boots (830) will be apparent to those of ordinary skill in the art.

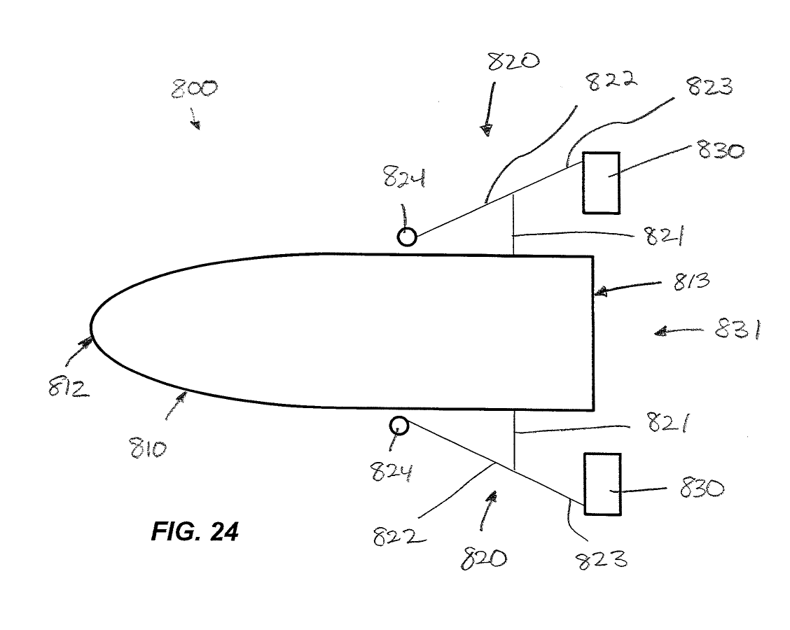

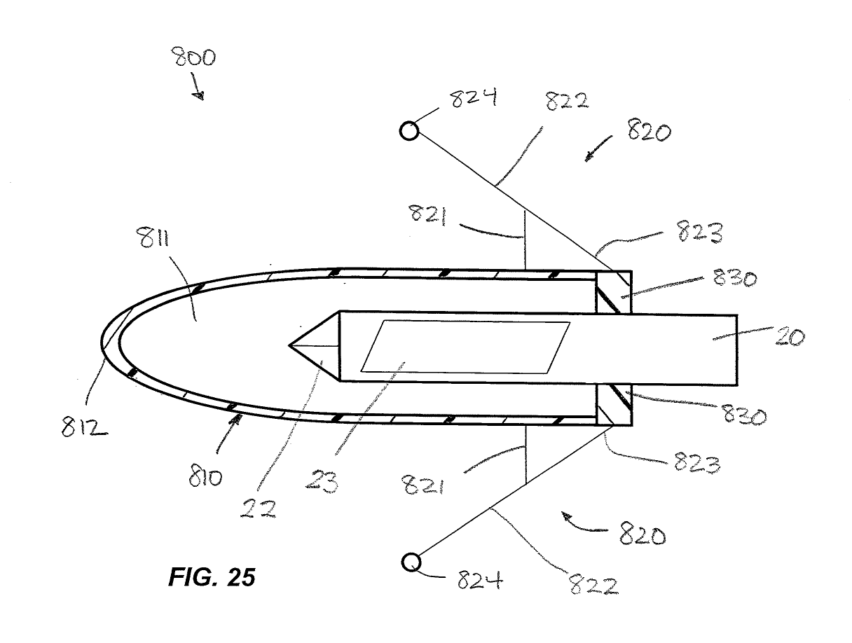

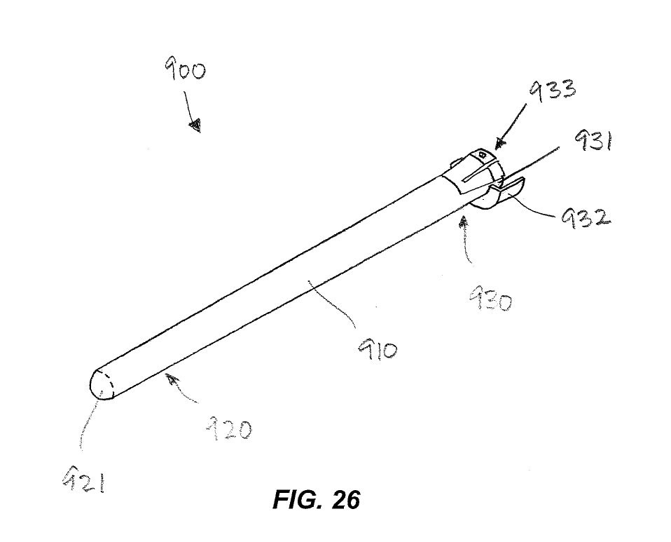

As shown in FIG. 23, when needle tip protector (800) is not installed on needle (20), living hinges (820) are in their initial or neutral position. In this position, elastomeric boots (830) are closest together. As shown in FIG. 24, in preparation for installation of needle tip protector (800) on needle (20), living hinges (820) are pressed inwardly near handlebars (824), thereby causing pivoting arms (822) to pivot and drive elastomeric boots (830) in an outward direction from one another. In this position, open proximal end (813) is exposed and ready to receive needle (20). As shown in FIG. 25, once needle (20) has been sufficiently inserted within hollow interior (811) of body (810), living hinges (820) are released and living hinges (820) return to their initial or neutral position. The elastomeric nature of boots (830) is such that upon contacting the outer surface of needle (20), boots (830) compress to create a frictionally secure connection with needle (20). In some versions, boots (830) are configured to completely surround the outer circumference of needle (20) where boots (830) contact the outer surface of needle (20). In some such versions, boots (830) also contact each other when boots (830) are secured to needle (20) in order to substantially seal the distal end of needle (20). In such a configuration, lateral aperture (23) is substantially sealed within hollow interior (811) of body (810), and needle (20) can be primed with saline or another treatment fluid prior to being used in a procedure.

To remove needle tip protector (800) from needle (20), the reverse process from that described above may be used. For instance, living hinges (820) are pressed near handlebars (824) to cause pivoting arms (822) to pivot, thereby driving boots (830) outward to the position shown in FIG. 24. From this point either needle tip protector (800) is advanced distally, or needle (20) is retracted proximally, to remove needle tip protector (800) from needle (20).

In some versions of needle (20), the edges of lateral aperture (23) can be sharp such that sliding lateral aperture (23) past another object can cause skiving of material from the object into lateral aperture (23). With the above described configuration for needle tip protector (800), namely one where elastomeric boots (830) are operably configured to move outward from a central longitudinal axis of needle tip protector (800) when installing or removing needle tip protector (800) on needle (20), the risk of skiving material from needle tip protector (800) into lateral aperture (23) may be reduced. Of course, in some versions, needle (20) may still be installed and removed from needle tip protector (800) by simply sliding needle (20) past elastomeric boots (830). For instance, this may occur by only partially actuating living hinges (820) or, in some versions, not actuating living hinges (820) at all when installing and removing needle tip protector (800) on needle (20). By way of example only, in some versions, a user may refrain from pressing living hinges (820) to drive boots (830) outward when installing and removing needle (20). Instead, a user may simply align needle tip (22) with space (831) and advance needle (20) into hollow interior (811) of body (810). The contact created between the outer surface of needle (20) and boots (830) creates force on boots (830) that in part causes boots (830) to compress and in part actuates living hinges (820) to drive boots (830) outward from needle (20). Other suitable alternative versions, features, components, configurations, and functionalities of needle tip protector (800) will be apparent to those of ordinary skill in the art in view of the teachings herein.

3. Needle Hub Inner Lock

FIGS. 26-29 show another exemplary needle tip protector (900). In this example, needle tip protector (900) comprises a body (910) having a distal portion (920) with a closed distal end (921), and a proximal portion (930) with an open proximal end (931). Body (910) has a hollow interior extending along the length of body (910). Proximal portion (930) comprises a lower cup (932) and an upper section (933). Lower cup (932) is configured to fit below the outer lower surface of needle hub (940) and surround the lower half of needle hub (940). Accordingly, lower cup (932) is configured with a larger diameter than needle hub (940). Upper section (933) is configured with an increasing taper from the distal-most portion of upper section (933) to the proximal-most portion of upper section (933). Upper section (933) comprises dual slots (934) that extend along a substantial portion of upper section (933). Slots (934) divide upper section (933) into outer sections (935) and central section (936). Slots (934) are operably configured to allow central section (936) to resiliently flex. Central section (936) comprises a locking projection (937) that is operably configured to engage a recess (941) within needle hub (940) as described in greater detail below.

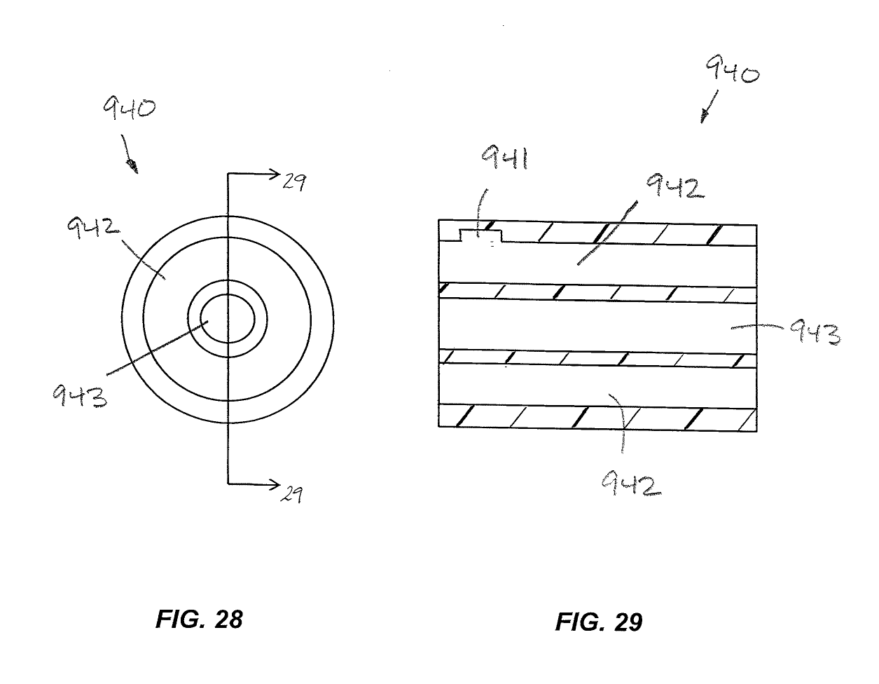

FIGS. 28-29 show an exemplary configuration of needle hub (940). In the present example, needle hub (940) comprises an outer chamber (942) and a recess (941). Needle hub (940) further comprises inner chamber (943), through which needle (20) is disposed. In the present example, outer chamber (942) and inner chamber (943) extend the length of needle hub (940). In some versions, outer chamber (942) extends only enough to permit locking projection (937) to engage recess (941). Based on the teachings herein, other configurations for needle hub (940), including its outer chamber (942), inner chamber (943), and recess (941) will be apparent to those of ordinary skill in the art.

In an exemplary use, needle tip protector (900) is used with a biopsy device (10) that is fitted with needle hub (940) and needle (20). Needle tip protector (900) is slid over needle (20), e.g. by holding needle (20) stationary and proximally advancing needle tip protector (900). Needle tip protector (900) is configured with a greater length than needle (20) such that proximal portion (930) of needle tip protector (900) contacts and ultimately engages needle hub (940), with needle tip (22) being spaced away from the distal-most part of the interior of needle tip protector (900) when needle tip protector (900) is engaged with needle hub (940). As proximal portion (930) approaches needle hub (940), proximal end (931) is aligned with an outer chamber (942) of needle hub (940) while lower cup (932) is aligned beneath the lower outer surface of needle hub (940). With the tapered configuration of proximal portion (930), and locking projection (937), the diameter of proximal portion (930) is slightly greater than that of the outer chamber (942) of needle hub (940). However, slots (934) allow central section (936) to flex inwardly, such that proximal end (931) of proximal portion (930) is able to fit within outer chamber (942). Once within outer chamber (942), locking projection (937) slides proximally within needle hub (940) until reaching recess (941), at which point locking projection (937) engages recess (941) due to its outward bias to return to its original, un-flexed, position. This engagement provides a secure, yet removable, connection between needle tip protector (900) and needle hub (940) such that needle (20) is protected.

After installation, to remove needle tip protector (900), a user presses central section (936), thereby causing central section (936) of proximal portion (930) to flex inward. This flexing action causes locking projection (937) to disengage recess (941). With locking projection (937) disengaged from recess (941), the user advances needle tip protector (900) distally such that proximal end (931) of proximal portion (930) is removed from needle hub (940). From this point, needle tip protector (900) is further advanced distally until needle (20) is completely removed from the hollow interior of body (910). Other suitable alternative versions, features, components, configurations, and functionalities of needle tip protector (900) will be apparent to those of ordinary skill in the art in view of the teachings herein.

4. Outer Seal Tapered Trocar

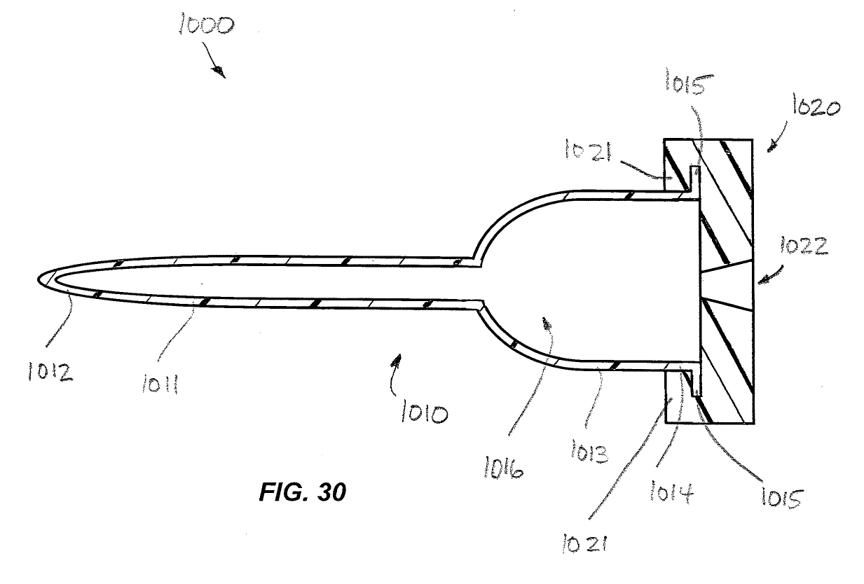

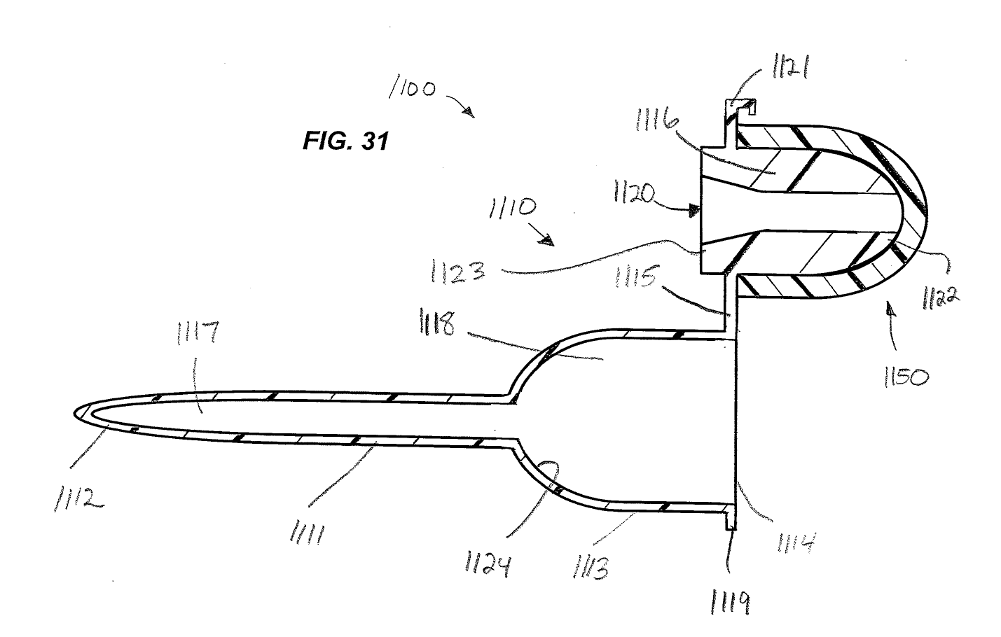

FIG. 30 shows another exemplary needle tip protector (1000). In this example, needle tip protector (1000) comprises a body (1010) and a seal (1020). In some versions, body (1010) is comprised of polycarbonate, however other suitable materials will be apparent to those of ordinary skill in the art in view of the teachings herein. Body (1010) comprises a distal portion (1011) having a closed distal end (1012); and proximal portion (1013) having an open proximal end (1014). Proximal portion (1013) comprises an outwardly extending annular flange (1015) about proximal end (1014). In some versions, body (1010) is configured to cover only enough of needle (20) such that lateral aperture (23) is contained within body (1010). In some such versions, with the accompaniment of seal (1020) as described below, needle (20) can be primed with saline or another treatment fluid before performing a procedure. Of course, body (1010) may be configured to cover more or less of needle (20) in other versions. In the present example, the polycarbonate of body (1010) is clear such that a user may see through body (1010). It should be understood, though, that body (1010) may be translucent or opaque and/or have any suitable combination of such properties.

In the present example, seal (1020) is comprised of an elastomeric material. Seal (1020) comprises hook members (1021) and opening (1022). Hook members (1021) are configured to engage flange (1015) of body (1010) to securely connect seal (1020) to body (1010). In some versions, seal (1020) surrounds the entire circumference of proximal end (1014) such that all of flange (1015) appears buried within seal (1020). In some other versions, seal (1020) surrounds a portion of the circumference of proximal end (1014) such that only a portion of flange (1015) appears buried within seal (1020). In some versions, seal (1020) is overmolded onto proximal end (1014) of body (1010); while in some other versions, seal (1020) is formed separately and then added onto proximal end (1014) of body (1010). Opening (1022) in seal (1020) is tapered to create a compressive fit with needle (20) when needle (20) is inserted through opening (1022). Opening (1022) further aligns with distal portion (1011), specifically hollow interior (1016) of body (1010) that coincides with the interior of distal portion (1011). Thus, when needle (20) is inserted through opening (1022), needle (20) may continue to be advanced distally without contacting the interior walls of body (1010). In the present example, distal end (1012) of body (1010) is configured such that the interior portions of distal end (1012) only contact the sides of needle tip (22) and not the sharp cutting edge of needle tip (22). In some versions, this is accomplished by using a core pin or some other support structure on the mold that resembles a diamond shape, thus keeping the contact points on the side of the needle tip (22) and not directly against the sharp cutting edge. In other words, the interior of distal end (1012) may provide a support structure that is perpendicular to the cutting plane of needle tip (22) to prevent skiving and keep needle tip (22) sharp. Of course, distal end (1012) may be formed in any other suitable fashion and may have any other suitable configuration.

In an exemplary use, a user aligns needle tip (22) of needle (20) with opening (1022) of seal (1020) and advances needle (20) distally, or advances needle tip protector (1000) proximally such that needle tip (22) slides past opening (1022) and into hollow interior (1016) of body (1010). Opening (1022) of seal (1020) is configured such that its narrowest point defines an inner diameter that is smaller than the outer diameter of needle (20). With this configuration, seal (1020) will compress against the outer surface of needle (20) due to its elastomeric properties, thereby creating a frictionally secure and sealed connection between needle (20) and needle tip protector (1000). Once needle (20) has been protected by needle tip protector (1000), needle (20) may be primed with saline or another treatment fluid since lateral aperture (23) is sealed within body (1010). To remove needle tip protector (1000), needle (20) would be advanced proximally relative to needle tip protector (1000) until needle tip (22) clears seal (1020). In some versions, to remove needle tip protector (1000), hook members (1021) are grasped and flexed outward such that hook members (1021) disengage flange (1015). At this point the combined needle (20) and seal (1020) are removed from body (1010). Needle (20) may then be removed from seal (1020) by sliding seal (1020) off needle (20), cutting seal (1020), etc. Other suitable alternative versions, features, components, configurations, and functionalities of needle tip protector (1000) will be apparent to those of ordinary skill in the art in view of the teachings herein.

5. Inner Seal Hinged Tapered Trocar

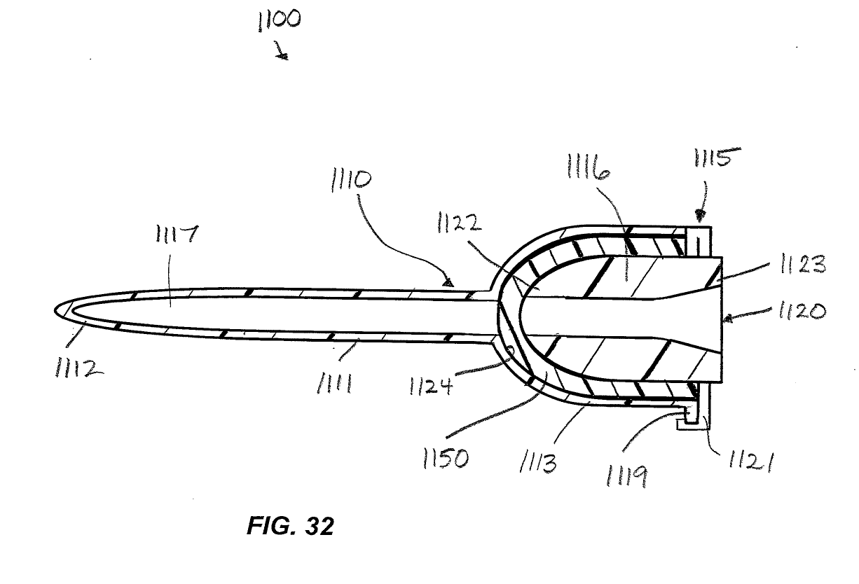

FIGS. 31-32 show another exemplary needle tip protector (1100). In the present example, needle tip protector (1100) comprises a body (1110) and a seal (1150). Body (1110) is constructed from a plastic material and comprises distal portion (1111) with closed distal end (1112), proximal portion (1113) with open proximal end (1114), living hinge (1115), and funnel section (1116). Distal portion (1111) of body (1110) includes hollow interior (1117). Proximal portion (1113) of body (1110) comprises hollow interior (1118), interior wall (1124), and locking member (1119) along a portion of proximal end (1114). In the present example, proximal portion (1113) has a conical shape such that hollow interior (1118) has a similar conical shape. Of course, proximal portion (1113) may have other shapes. Living hinge (1115) connects proximal end (1114), opposite locking member (1119), with funnel section (1116). Living hinge (1115) is operably configured to permit rotation of funnel section (1116) approximately 180 degrees, as shown in comparing FIG. 31 with FIG. 32.

In the present example, funnel section (1116) is configured with a profile that approximates the shape and volume of hollow interior (1118). However, funnel section (1116) alone does not fill hollow interior (1118), but the combination of funnel section (1116) and seal (1150) does fill hollow interior (1118). It should be understood that completely filling hollow interior (1118) is by no means necessary in all versions. Funnel section (1116) comprises passage (1120), which has a tapered configuration, latch (1121), first end (1122), and second end (1123). When funnel section (1116) is located within hollow interior (1118) of proximal portion (1113) as shown in FIG. 32, passage (1120) is configured to align with hollow interior (1117) of distal portion (1111). Latch (1121) is also configured to make a snap connection with locking member (1119) of proximal portion (1113). Other alternative types of connections for latch (1121) and locking member (1119) will be apparent to those of ordinary skill in the art in view of the teachings herein.

In the present example, seal (1150) comprises an elastomeric material located along first end (1122) of funnel section (1116). When funnel section (1116) and seal (1150) are (1113) positioned within hollow interior (1118) of proximal portion (1113), as shown in FIG. 32, seal (1150) has sufficient resilient properties to create a compressive seal between first end (1122) of funnel section (1116) and interior wall (1124) of proximal portion (1113). In the present example, seal (1150) is operably configured as a self-sealing component where no opening in seal (1150) is provided, except for that created by needle tip (22) when needle tip (22) is passed through seal (1150) during installation of needle tip protector (1100) over a portion of needle (20) as discussed further below. In some other versions, seal (1150) may be configured with a slit or a pre-defined opening for receiving needle (20).

In some versions, body (1110) is constructed as a single piece that includes distal portion (1111) with closed distal end (1112), proximal portion (1113) with open proximal end (1114), living hinge (1115), and funnel section (1116). Seal (1150) is constructed as a separate piece, and could be placed on funnel section (1116), as shown, by a user or by the manufacturer of needle tip protector (1100). In an exemplary use, with seal (1150) in place, funnel section (1116) is rotated approximately 180 degrees via living hinge (1115) such that funnel section (1116) is positioned within hollow interior (1118) of proximal portion (1113). Needle (20) is then inserted within and through passage (1120) such that needle tip (22) pierces or otherwise traverses seal (1150), which then seals around the outer surface of needle (20). In the present example, needle (20) is advanced toward distal end (1112) until needle tip (22) and lateral aperture (23) are distal of seal (1150). With this configuration, needle (20) may be primed with saline or another treatment fluid in advance of performing a procedure. To remove needle tip protector (1100) needle (20) is retracted proximally until needle tip (22) is clear of passage (1120). After completing a procedure, needle (20) may be reinserted within needle tip protector (1100) in the same manner as described above. In some versions, seal (1150) is easily removed and replaceable thereby extending the useful life of needle tip protector (1100). Other suitable alternative versions, features, components, configurations, and functionalities of needle tip protector (1100) will be apparent to those of ordinary skill in the art in view of the teachings herein.

C. Exemplary Integral Needle Tip Protectors

Some needle tip protectors may be configured as integral or built-in components of a biopsy device (10). Such needle tip protectors can be selectively actuated to extend over and protect the needle tip (22) from damage during handling, and/or protect a user from accidental contact with the sharp needle tip (22). Such needle tip protectors can further be selectively actuated to be withdrawn from the area surrounding the needle (20) such that a procedure can be performed. Several examples of such exemplary needle tip protectors are described in the paragraphs that follow, while several other examples will be apparent to those of ordinary skill in the art in view of the teachings herein.

1. Extending Hub

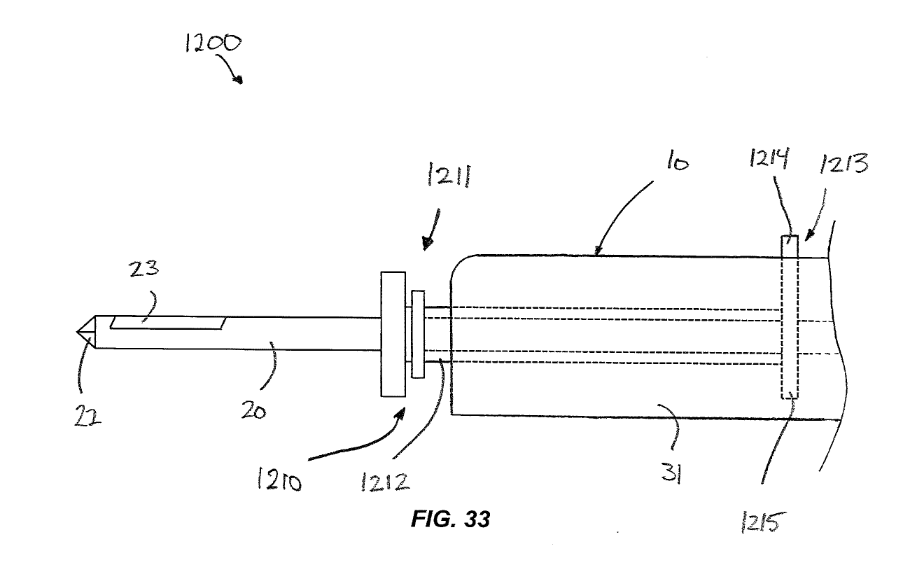

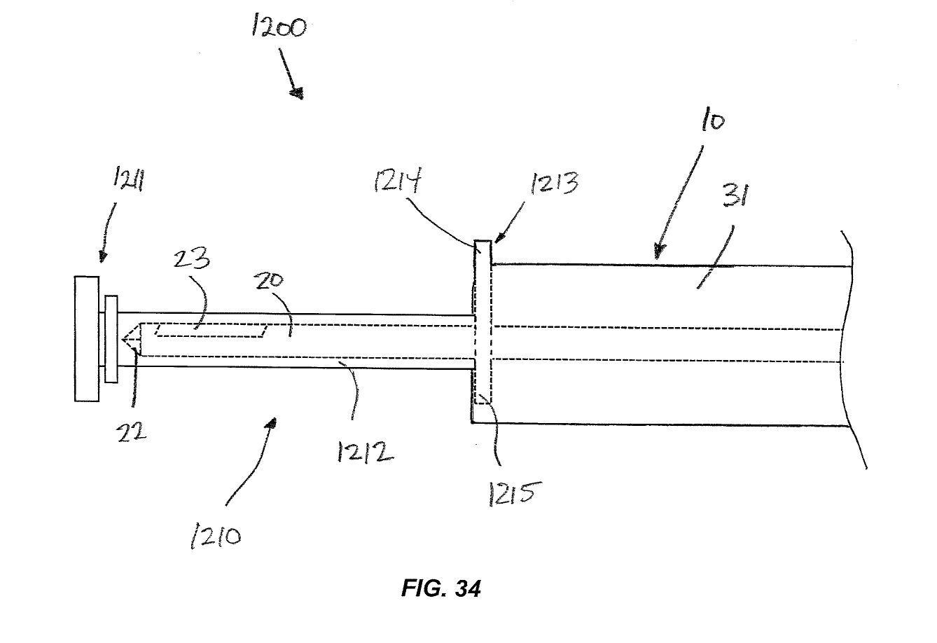

FIGS. 33-34 show another exemplary needle tip protector (1200). Needle tip protector (1200) of this example is configured as a built-in, integral component of biopsy device (10). Needle tip protector (1200) comprises an elongated needle hub (1210), which is used in place of needle hub (24) described above. Elongated needle hub (1210) comprises a distal portion (1211), a cover portion (1212), and an actuator (1213). Cover portion (1212) of elongated needle hub (1210) has a hollow tube structure that it is configured to surround the outer diameter of needle (20). As shown in the comparison of FIGS. 33 and 34, elongated needle hub (1210) is operably configured to extend distally from within housing (31) of biopsy device (10) to cover needle (20). Also, when not covering needle (20), elongated needle hub (1210) is operably configured to partially retract proximally within housing (31).

In the present example, biopsy device (10) is initially provided with cover portion (1210) in a distal, extended position to cover needle (20). At the beginning of a biopsy procedure, actuator (1213) is manually pushed or pulled proximally by the user to retract cover portion (1210), thereby revealing needle (20). After the biopsy procedure is complete, actuator (1213) is manually pushed distally by the user to re-cover needle (20). As shown, actuator (1213) includes a first end (1214) and a second end (1215). First end (1214) protrudes from a track or slot (not shown) in housing (31) such that a user has access to actuator (1213). The action of manually pushing actuator (1213) distally correspondingly drives elongated needle hub (1210) distally such that it can extend and cover needle (20). In the present example, actuator (1213) is manually driven distally until actuator (1213) contacts the distal end of housing (31), at which point the track or slot guiding actuator (1213) terminates. Of course, in some other versions, the track or slot may be configured to terminate proximally from the distal end of housing (31), and elongated needle hub (1210) may be configured to still be of sufficient length to fully cover needle (20).

A variety of structures may be used with elongated needle hub (1210) to keep elongated needle hub (1210) aligned with needle (20) and also to support elongated needle hub (1210). For example, the protruding actuator (1213) and track or slot (not shown) through which actuator (1213) protrudes may operate to guide elongated needle hub (1210). To provide balanced support and guide elongated needle hub (1210), housing (31) may include an internal groove (not shown) opposite the track or slot that retains second end (1215) of actuator (1213). In this configuration, elongated needle hub (1210) is supported and guided on at least two opposing sides. Various other structures and modifications to elongated needle hub (1210) and biopsy device (10) to support and guide elongated needle hub (1210) will be apparent to those of ordinary skill in the art in view of the teachings herein.

In some versions, the extension and retraction of elongated needle hub (1210) may use similar actuation to that of a switch-blade style knife. For instance, biopsy device (10) and elongated needle hub (1210) may be configured with a system of springs or spring-like structures such that elongated needle hub (1210) may be extended and/or retracted with a push button. In some such versions, one or more safeties or lock-outs may be incorporated such as to prevent inadvertent extension or retraction of elongated needle hub (1210). Similarly, biopsy device (10) and elongated needle hub (1210) may be configured with a lock-out structure that substantially prevents retraction of elongated needle hub (1210) once needle hub (1210) has been extended. In some such versions, biopsy device (10) and elongated needle hub (1210) may be configured for one time actuation after a procedure has been completed. Still in other versions, biopsy device (10) and elongated needle hub (1210) may be configured to include a lock-out that may be selectively disengaged such that elongated needle hub (1210) could be retracted after being extended. Other alterations and functionalities involving lock-out structures will be apparent to those of ordinary skill in the art in view of the teachings herein. Furthermore, various suitable ways in which such safety and/or lock-out systems may be configured and operable will be understood by those or ordinary skill in the art in view of the teachings herein.

While several of the above examples have described elongated needle hub (1210) as being used to protect needle tip (22) after a procedure has been completed, elongated needle hub (1210) may also be configured to protect needle tip (22) in advance of a procedure. For example, in a biopsy device (10) incorporated elongated needle hub (1210), the manufacturer may actuate elongated needle hub (1210) prior to packaging and shipping biopsy device (10). Then, a user may retract elongated needle hub (1210) prior to a procedure, and then re-extend elongated needle hub (1210) once the procedure is complete. Other suitable alternative versions, features, components, configurations, and functionalities of needle tip protector (1200) will be apparent to those of ordinary skill in the art in view of the teachings herein.

2. Extending Shield

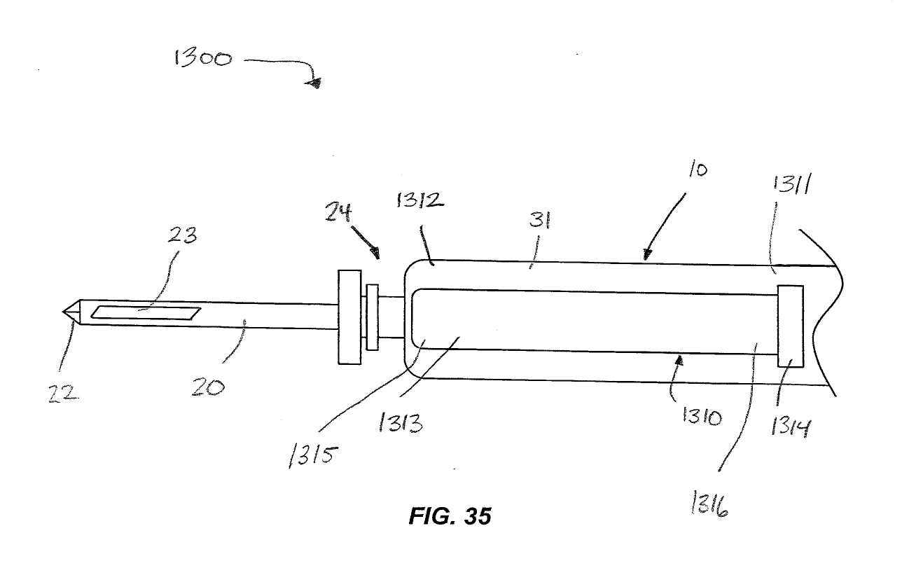

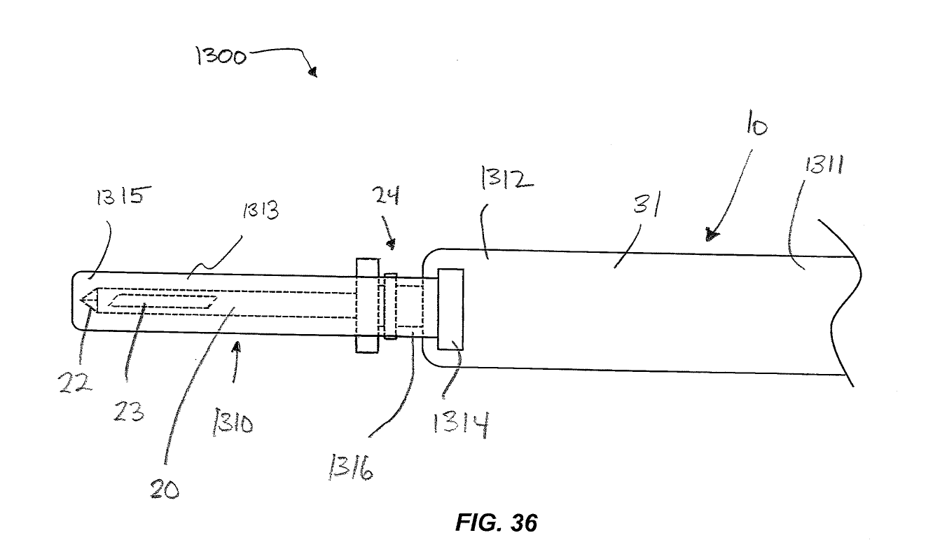

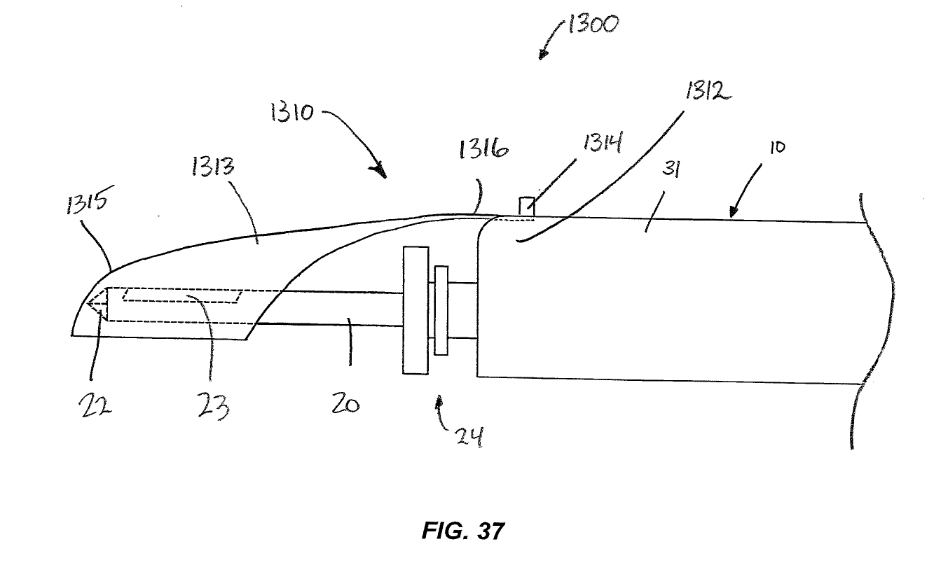

FIGS. 35-37 show another exemplary needle tip protector (1300). Needle tip protector (1300) of this example is configured as a built-in, integral structure to biopsy device (10). Needle tip protector (1300) comprises shield (1310) that is a component of housing (31) of biopsy device (10). In the initial position shown in FIG. 35, shield (1310) is positioned along an upper surface of housing (31), extending from a proximal region (1311) of housing (31) to a distal region (1312) of housing (31). In the extended position shown in FIGS. 36-37, shield (1310) extends from housing (31) to cover needle (20). Specific features and uses of shield (1310) and needle tip protector (1300) are discussed further below.

Shield (1310) of the present example comprises a cover (1313) and a tab actuator (1314). Cover (1313) includes a distal portion (1315) and a proximal portion (1316). In the present example, cover (1313) of shield (1310) rests immediately adjacent to housing (31) when needle tip protector (1300) is in the initial position shown in FIG. 5. In this position, cover (1313) assumes a relatively flat shape that has a lateral extending crown matching the profile of the upper surface of housing (31). The material properties of cover (1313) hold cover (1313) against housing (31). More specifically, cover (1313) is constructed of a flexible, yet strong material and cover (1313) has a natural curved shape as shown in the side view of FIG. 37. When cover (1313) is in the retracted position such that cover (1313) is adjacent housing (31), cover (1313) is flexed to conform to the outer profile shape of housing (31). The natural curved shape of cover (1313), and cover (1313) being retained in a flexed state, causes cover (1313) to return to its curved shape when cover (1313) is extended distally as shown in FIG. 37. Of course when cover (1313) is not extended cover (1313) may be retained adjacent housing (31) by using other suitable means. For example, in some versions, housing (31) may be configured with an upper slot (not shown) extending along the upper surface of housing (31). Such an upper slot would hold cover (1313) against housing (31) when cover (1313) is not extended. Still other alterations and functionalities for cover (1315) will be apparent to those of ordinary skill in the art based on the teachings herein.

Tab actuator (1314) is connected with proximal portion (1316) of cover (1313). In the present example, tab actuator (1314) is used in manually advancing cover (1313) in a distal direction when wanting to protect needle tip (22) of needle (20). Tab actuator (1314) resides in a track or slot similar to the discussion above with respect to FIGS. 33-34. In some versions, tab actuator (1314) functions as a release actuator that releases cover (1313) such that cover (1313) extends distally automatically upon pressing tab actuator (1314). For example, in some versions needle tip protector (1300) is spring loaded and locked into the position as shown in FIG. 35 by the manufacturer. In some such versions, tab actuator (1314) is configured to unlock needle tip protector (1300) by releasing the lock (not shown) that holds the spring (not shown) that maintains needle tip protector (1300) in the loaded position. Such a releasing action causes the spring (not shown) to unload and drive cover (1313) distally to extend over needle tip (22) of needle (20).

In versions of biopsy device (10) having needle tip protector (1300) configured with a system of springs or spring-like structures to deploy cover (1313) to an extended position, one or more safeties or lock-outs may be incorporated to substantially prevent inadvertent extension or retraction of cover (1313). Similarly, needle tip protector (1300) may be configured with a lock-out structure that substantially prevents retraction of cover (1313) once cover (1313) has been extended. In some such versions, biopsy device (10) and needle tip protector (1300) may be configured for one time actuation after a procedure has been completed. Still in other versions, biopsy device (10) and needle tip protector (1300) may be configured to include a lock-out that may be selectively disengaged such that cover (1313) could be retracted after being extended. Other alterations and functionalities involving lock-out structures will be apparent to those of ordinary skill in the art in view of the teachings herein. Furthermore, various suitable ways in which such safety and/or lock-out systems may be configured and operable will be understood by those or ordinary skill in the art in view of the teachings herein. Still other alterations and functionalities for tab actuator (1314) will be apparent to those of ordinary skill in the art based on the teachings herein.

While several of the above examples have described needle tip protector (1300) as being used to protect needle tip (22) after a procedure has been completed, needle tip protector (1300) may also be configured to protect needle tip (22) in advance of a procedure. For example, in a biopsy device (10) incorporating needle tip protector (1300), the manufacturer may extend cover (1313) prior to packaging and shipping biopsy device (10). Then a user may retract cover (1313) prior to a procedure, and then re-extend cover (1313) once the procedure is complete. Other suitable alternative versions, features, components, configurations, and functionalities of needle tip protector (1300) will be apparent to those of ordinary skill in the art in view of the teachings herein.

Several of the needle tip protectors disclosed herein include components constructed from elastomeric materials. Any of several elastomeric materials may be suitable for use with any such needle tip protector that includes elastomeric components. By way of example only, suitable elastomeric materials include thermosetting plastics that may require vulcanization, thermoplastic elastomers (e.g. Santoprene™ among others), natural rubber, synthetic rubbers (e.g., ethylene propylene diene M-class—EPDM—among others), and other polymers having suitable elastic properties. Several of the needle tip protectors disclosed herein include components constructed from plastic non-compressible materials. Any of several plastics may be suitable for use with any such needle tip protector that includes plastic non-compressible components. By way of example only, suitable plastic materials include medical grade thermoplastics and thermosetting polymers (e.g., polycarbonate, polyethylene, polystyrene, polyvinyl chloride and polytetrafluoroethylene (PTFE) and others). Other suitable alternative materials for the above elastomerics and plastics will be apparent to those of ordinary skill in the art in view of the teachings herein.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the present invention have application in conventional endoscopic and open surgical instrumentation as well as application in robotic-assisted surgery.

Embodiments of the devices disclosed herein can be designed to be disposed of after a single use, or they can be designed to be used multiple times. Embodiments may, in either or both cases, be reconditioned for reuse after at least one use. Reconditioning may include any combination of the steps of disassembly of the device, followed by cleaning or replacement of particular pieces, and subsequent reassembly. In particular, embodiments of the device may be disassembled, and any number of the particular pieces or parts of the device may be selectively replaced or removed in any combination. Upon cleaning and/or replacement of particular parts, embodiments of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/we claim:

1. A device for use in a biopsy procedure, wherein the device comprises:
a. a body portion, wherein the body portion comprises a housing;
b. a hub, wherein the hub is coupled with the housing;
c. a needle, wherein the needle extends distally from the hub, wherein the needle comprises:
i. an outer cannula defining an inner lumen,
ii. a tissue piercing tip, and
iii. a lateral aperture configured to receive a tissue specimen;
d. a cutter comprising a distal cutting edge, wherein the cutter is operably configured to translate relative to the needle to sever the tissue specimen received within the lateral aperture; and
e. a needle tip protector, the needle tip protector comprising a cover portion configured to shield the tissue piercing tip, wherein the needle tip protector is detachable from the device.

2. The device of claim 1, wherein the cover portion comprises a closed distal end.

3. The device of claim 1, wherein the cover portion comprises an open distal end.

4. The device of claim 1, wherein the needle tip protector comprises an elastomeric connector, wherein the elastomeric connector is operably configured to secure the needle tip protector to the needle.

5. The device of claim 4, wherein the elastomeric connector is configured to provide a fluid-tight seal of the lateral aperture of the needle.

6. The device of claim 1, wherein the needle tip protector comprises an elastomeric seal, wherein the elastomeric seal provides a fluid-tight seal of the lateral aperture of the needle.

7. The device of claim 6, wherein the elastomeric seal seals against the hub.

8. The device of claim 1, wherein the needle tip protector is constructed at least in part from an elastomeric material.

9. The device of claim 1, wherein the needle tip protector comprises a living hinge, wherein the living hinge is operably configured to selectively attach the needle protector to the device.

10. The device of claim 9, wherein the needle tip protector comprises an elastomeric seal, wherein the living hinge and the elastomeric seal are configured to provide a fluid-tight seal of the lateral aperture of the needle.

11. The device of claim 9, wherein the living hinge comprises a locking projection configured to engage a locking recess in the hub.

12. The device of claim 1, wherein the needle tip protector comprises a living hinge, wherein the living hinge is operably configured to actuate the cover of the needle tip protector to shield or expose the tissue piercing tip of the needle.

13. The device of claim 12, wherein the living hinge actuates the cover by selectively extending and retracting the cover.

14. The device of claim 12, wherein the cover comprises a first half and a second half, wherein the living hinge is operable to actuate the cover by moving the first half and the second half together to shield the tissue piercing tip of the needle.

15. The device of claim 1, wherein the needle tip protector comprises a connector, wherein the connector is operably configured to snap-fit the needle tip protector to the device.

16. The device of claim 15, wherein the connector is operably configured to snap-fit to the hub.

17. The device of claim 15, wherein the connector is operably configured to snap-fit to the housing.

18. The device of claim 15, wherein the connector is operably configured to snap-fit to the needle.

19. A device for use in a biopsy procedure, wherein the device comprises:
a. a body portion, wherein the body portion comprises a housing;
b. a hub, wherein the hub is coupled with the housing;
c. a needle, wherein the needle extends distally from the hub, wherein the needle comprises:
i. an outer cannula defining an inner lumen,
ii. a tissue piercing tip, and
iii. a lateral aperture configured to receive a tissue specimen;
d. a cutter comprising a distal cutting edge, wherein the cutter is operably configured to translate relative to the needle to sever the tissue specimen received within the lateral aperture; and
e. a needle tip protector, the needle tip protector comprising a cover portion configured to shield the tissue piercing tip, wherein the needle tip protector is selectively coupled with the device, wherein the needle tip protector is operably configured to selectively couple with the device from a position laterally adjacent to the device.

20. A detachable tip protector for use with a biopsy device, the biopsy device comprising a body portion having a housing, a hub connected to the housing, a needle extending from the hub, the needle comprising an outer cannula defining an inner lumen, a tissue piercing tip, and a lateral aperture configured to receive a tissue specimen, the biopsy device further comprising a cutter comprising a distal cutting edge, wherein the cutter is operably configured to translate relative to the needle to sever the tissue specimen received within the lateral aperture, wherein the tip protector comprises:
a. a cover portion configured to shield the tissue piercing tip on at least two sides, wherein the cover portion is actuated by a living hinge to shield and expose the tissue piercing tip;
b. a seal configured to fluidly seal the lateral aperture of the needle, wherein the seal comprises an elastomeric material; and
c. a connector configured to selectively secure the tip protector to the biopsy device.
